(19) United States
(12) Patent Application Publication
(10) Pub. No.: US 2022/0196661 A1
(43) Pub. Date: Jun. 23, 2022

(54) Cardiomyocyte-Specific Biological Markers and Uses Thereof

(71) Applicant: Rebekah L. Gundry, Milwaukee, WI (US)

(72) Inventor: Rebekah L. Gundry, Milwaukee, WI (US)

(21) Appl. No.: 17/127,237

(22) Filed: Dec. 18, 2020

(51) Int. Cl. G01N 33/577; C07K 16/28; G01N 33/569

(52) U.S. Cl. CPC G01N 33/577; C07K 16/28; G01N 2800/32; C07K 2317/34; G01N 33/56966

(57) ABSTRACT

The present invention is directed to particular monoclonal antibodies and fragments thereof that find use in the detection and isolation of in vitro differentiated cardiomyocytes. In particular, the present invention relates to LSMEM2 biomarkers, including monoclonal antibodies having specificity for cell surface protein LSMEM2 and to methods of using such biomarkers for diagnosis of cardiovascular injury.

### CROSS-REFERENCE TO RELATED APPLICATIONS

This application is a continuation application to U.S. application Ser. No. 15/739,292 filed on Dec. 22, 2017, which was a 371 of international Application No. PCTUS2016038637, filed on Jun. 22, 2016, which claims priority to U.S. Provisional Patent Application No. 62/184,397, filed Jun. 25, 2015, all of which are incorporated herein by reference in their entireties.

### STATEMENT REGARDING FEDERALLY FUNDED RESEARCH OR DEVELOPMENT

This invention was made with government support under grant number R00-HL094708 awarded by the National Institutes of Health. The government has certain rights in the invention.

### BACKGROUND

Adult onset diseases of the cardiovascular system, congenital heart disease, and heart failure are major health risks throughout the industrialized world. Due to the mortality and morbidity associated with such diseases, there is great interest in finding effective therapeutic methods for repairing damaged heart tissue, methods for regenerating cardiomyocytes, and clinically relevant models of cardiac disease. Cell transplantation has emerged as a therapeutic approach to increasing the number of contractile myocytes available for the repair of damaged hearts. Human induced pluripotent stem (human iPS) cells are a viable source for in vitro generation of cardiomyocytes (iCMs) that are useful for the study of human development, heart disease modeling, and regenerative medicine. While antibodies having specificity for various cell-surface markers on human pluripotent stem cell-derived cells can be used to identify and isolate populations in sufficient numbers for further study (Dubois et al., Nature Biotech. 29:1011-18 (2011)), levels of biomarker expression vary with the stage of cardiomyocyte differentiation and nodal, atrial, and ventricular cardiomyocytes have different gene and protein expression profiles (Ng et al., Biomaterials 32(30):7571-80 (2011)).

The inability to reliably identify heart chamber-specific cardiomyocytes of a specific maturation stage in a high-throughput fashion has been a barrier to utilizing such cells for regenerative medicine. Accordingly, there remains a need in the art for a clinically relevant system for identifying and isolating stage- and subtype-specific cardiomyocytes having defined functional properties. There also remains a need in the art for subtype-specific biomarkers useful for predicting cardiac disease and identifying candidates for specific therapies.

### SUMMARY OF THE INVENTION

In one embodiment, the present invention is directed to particular monoclonal antibodies and fragments thereof that find use in the detection and isolation of in vitro differentiated cardiomyocytes. In particular, the present invention relates to LSMEM2 biomarkers, including monoclonal antibodies having specificity for cell surface protein LSMEM2 and to methods of using such biomarkers for diagnosis of cardiovascular injury.

In a first aspect, provided herein is a monoclonal antibody or antigen binding fragment thereof that is capable of binding an epitope of a LSMEM2 polypeptide having the amino acid sequence of SEQ ID NO:2. The epitope can comprise an amino acid sequence selected from the group consisting of SEQ ID NO:4, SEQ ID NO:5, SEQ ID NO:6, SEQ ID NO:7, and SEQ ID NO:8. The epitope can consist of an amino acid sequence selected from the group consisting of SEQ ID NO:4, SEQ ID NO:5, SEQ ID NO:6, SEQ ID NO:7, and SEQ ID NO:8. The monoclonal antibody can be selected from the group consisting of a chimeric antibody, a recombinant IgG (rIgG) antibody, a diabody, a single chain antibody, a multispecific antibody, and a humanized antibody. The antigen binding fragment can be selected from the group consisting of a Fab, a Fab′, a F(ab′)2, and a Fv.

In another aspect, provided herein is a method for isolating human cardiomyocytes from a cell mixture derived from human pluripotent stem cells. The method comprises or consists essentially of contacting to the cell mixture a monoclonal antibody or antigen binding fragment thereof as provided herein, and recovering antibody-bound cells from the contacted cell mixture, where the recovered cell population is a substantially homogeneous population of human cardiomyocytes.

In a further aspect, provided herein is a method for separating a cell population containing human cardiomyocytes from a cell mixture derived from human pluripotent stem cells. The method comprises or consists essentially of contacting to a cell mixture the monoclonal antibody or antigen binding fragment thereof as provided herein, and separating antibody-bound cells from the contacted cell mixture, where the separated cell population is a substantially homogeneous population of human cardiomyocytes.

In another aspect, provided herein is a method for detecting human cardiomyocytes in a cell mixture from human pluripotent stem cells. The method comprises or consists essentially of contacting to the cell mixture the monoclonal antibody or antigen binding fragment thereof as provided herein; and detecting the antibody.

In another aspect, provided herein is a method for producing a monoclonal antibody that binds specifically to LSMEM2, the method comprising or consisting essentially of (a) providing a peptide having an amino acid sequence selected from the group consisting of SEQ ID NO:4, SEQ ID NO:5, SEQ ID NO:6, SEQ ID NO:7, and SEQ ID NO:8; (b) administering the peptide to a mammal under conditions appropriate for the stimulation of an immune response; (c) isolating antibody producing cells from the mammal; (d) fusing the antibody producing cells with immortalizing cells to produce a hybridoma cell line; and (e) screening the hybridoma cell line to identify cell lines secreting monoclonal antibody that binds specifically to LSMEM2. In some cases, the peptide is produced synthetically.

In a further aspect, provided herein is a method for predicting, diagnosing, or monitoring a cardiovascular injury in a subject, the method comprising or consisting essentially of measuring the level of expression of LSMEM2 in a biological sample obtained from the subject; and comparing the level of LSMEM2 with the LSMEM2 level from a control sample, wherein a measured level or characteristic of LSMEM2 that is different than the control level or characteristic is indicative of a cardiovascular injury.

The biological sample can be selected from the group consisting of whole blood, a blood fraction, plasma, serum, urine, and heart tissue sample. The cardiovascular injury can be selected from the group consisting of myocardial infarction, cardiac ischemia, acute coronary syndrome, cardiomyopathy, heart failure, cardiac remodeling, and cardiac dilation. Measuring can comprise contacting a LSMEM2 binding agent to the sample; and (b) measuring the level of LSMEM2 bound to the binding agent. The binding agent can be an antibody or antigen-binding fragment thereof. The binding agent can be a monoclonal antibody or fragment thereof that is capable of binding an epitope of a LSMEM2 polypeptide having the amino acid sequence of SEQ ID NO:2. The antibody or antigen-binding fragment thereof can be immobilized on a solid support. The level of LSMEM2 can be measured using an assay selected from RIA, ELISA, mass spectroscopy, fluoroimmunoassay, immunofluorometric assay, immunoradiometric assay.

In another aspect, provided herein is a method for assessing susceptibility to a condition associated with cardiovascular injury in a subject, the method comprising or consisting essentially of measuring the level of expression of LSMEM2 in a biological sample obtained from the subject; and comparing the level of LSMEM2 with the LSMEM2 level from a control sample, wherein a measured level or characteristic of LSMEM2 that is different than the control level or characteristic is indicative of increased susceptibility to cardiovascular injury.

In yet another aspect, provided herein is a kit comprising a LSMEM2 biomarker and instructions for using the biomarker for evaluating the presence or occurrence of a cardiovascular injury. The biomarker can be a monoclonal antibody or antigen binding fragment thereof that is capable of binding an epitope of a LSMEM2 polypeptide comprising the amino acid sequence of SEQ ID NO:2. Expression of the biomarker in a biological sample can be determined using components of the kit. The biological sample can be selected from the group consisting of whole blood, a blood fraction, plasma, and heart tissue. The kit can be used to obtain a biomarker profile. The kit optionally comprises at least one internal standard useful to obtain the biomarker profile.

These and other features, aspects, and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents and alternatives. Reference should therefore be made to the claims recited herein for interpreting the scope of the invention.

### BRIEF DESCRIPTION OF THE DRAWINGS

The following drawings form part of the present specification and are included to further demonstrate certain aspects of the compositions and methods provided herein. The invention may be better understood by reference to one or more of these drawings in combination with the detailed description of specific embodiments presented herein.

FIGS. 1A-1C demonstrate transcriptional analysis of LSMEM2 during in vitro differentiation and in tissues. (A) qRT-PCR analysis of LSMEM2, and an intracellular reference marker of cardiomyogenesis (troponin I type 3; “TNNI3”), and an intracellular ventricle marker (Iroquois Homeobox Protein 4; “IRX4”) during the first 100 days of iCM differentiation (n=3). (B) qRT-PCR analysis of LSMEM2 and IRX4 in fetal sections (19 week hearts; 12 week skeletal muscle; n=2 for all samples) and non-diseased adult heart (average of 41 year old male and 22 year old female). Data normalized to right atria. Error bars=SEM. (C) Transcriptome sequencing (RNA-seq) data for LSMEM2 in 16 adult tissues (using the Illumina Human Body Map 2.0 RNA-Seq dataset).

FIG. 2 presents a sequence alignment of LSMEM2 protein among species. The predicted SH3 ligands in the human sequence are annotated by the black bars.

FIGS. 3A-3C present the amino acid sequences of (A) antigen sequences (epitopes) for nine monoclonal antibody clones and (B) LSMEM2. These monoclonal antibodies have specificity to the extracellular domain of LSMEM2 (as determined by CSC-Technology proteomics data). (C) Flow cytometry histograms for LSMEM2 (using Ruby 1 antibody), TNNI3 (cardiomyocyte marker), IRX4 (ventricular cardiomyocyte marker), MLC2a (early cardiomyocyte marker that in adult persists in the atrium), and MLC2v (ventricular cardiomyocyte marker) during the first 100 days of in vitro differentiation.

FIG. 4A-4D presents flow cytometric data for four different LSMEM2 mAbs (Ruby 1 (A), Ruby 2 (C), Ruby 4 (B), and Ruby 5 (D)).

FIGS. 5A-5B present data for day 10 iCM sorted by fluorescence activated cell sorting (FACS) data using LSMEM2 mAb #1 (“Ruby 1”). (A) Images of cells post-FACS demonstrate robust cell survival. (B) qRT-PCR analysis of sorted cells demonstrate that LSMEM2⁺ cells show enrichment of ventricular markers (IRX4, MLC2V) and depletion of atrial markers (COUP-TFII, NPPA, MLC2A) and immature cardiomyocyte markers (TNNI1, TNNI2, MYH11, MYOG). COUP-TFII=COUP transcription factor II (also known as NR2F2); NPPA=natriuretic peptide precursor A; TNNI1=troponin I type 3; TNNI2=troponin I type 2; MYH11=myosin, heavy polypeptide 11; MYOG=myogenin (also known as myogenic factor 4 or “MYF4”). n=3. Error bars=SEM.

FIGS. 6A-6B present images showing human (A) and mouse (B) adult ventricle tissue stained for LSMEM2. In A: (i) longitudinal right ventricle section; (ii) transverse left ventricle section co-stained with TNNT2 (green); and (iii) longitudinal right ventricle section shown in three-dimensional projection image. DNA=blue.

FIGS. 7A-7B present data related to the potential utility of LSMEM2 as a clinically relevant marker for cardiomyocyte death/health and/or heart disease. (A) qRT-PCR data for LSMEM2 and IRX4 (an intracellular ventricular marker) in left ventricle tissue samples from adult healthy (n=7) and failing heart (n=2). (B) Dot blot of human patient sera using Ruby 2 antibody to detect the extracellular form of LSMEM2, and anti-TNNI3 antibody to detect cardiac troponin. Image demonstrates that patients with cardiac ischemia (positive for cardiac troponin) also have detectable levels of LSMEM2 in serum.

FIG. 8 shows immunofluorescent staining of LSMEM2 using Ruby 2 antibody in human adult heart chambers, showing specific staining of the cardiomyocytes. Scale bar=50 μm.

FIGS. 9A-9C. Specificity of LSMEM2 antibodies. (A-B) show specificity of Ruby 2 for its peptide sequence as demonstrated by the elimination of antibody binding when excess peptide antigen is provided, as demonstrated by flow cytometry (A) and western blot (B). (C) Presence of Ruby 2 is co-incident with TNNI3, indicating that Ruby 2 is present on the cell surface of cardiomyocytes.

### DETAILED DESCRIPTION OF THE INVENTION

All publications, including but not limited to patents and patent applications, cited in this specification are herein incorporated by reference as though fully set forth in the present application.

The term “about” as used herein when referring to a measurable value such as a parameter, an amount, a temporal duration, and the like, is meant to encompass variations of and from the specified value, in particular variations of ±10% or less, preferably ±5% or less, more preferably ±1% or less, and still more preferably ±0.1% or less of and from the specified value, insofar such variations are appropriate to perform in the disclosed invention. It is to be understood that the value to which the modifier “about” refers is itself also specifically, and preferably, disclosed.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. Obviously, the term comprises also encompasses the closed wording “consisting of” as one of its embodiments.

The recitation of numerical ranges by endpoints includes all numbers and fractions subsumed within the respective ranges, as well as the recited endpoints.

Unless otherwise specified, all terms used in disclosing the invention, including technical and scientific terms, have the meaning as commonly understood by one of ordinary skill in the art to which this invention belongs. By means of further guidance, term definitions may be included to better appreciate the teaching of the present invention.

The present invention is based at least in part on the inventor's discovery of a cell surface marker (Leucine-rich Single-pass Membrane Protein 2 (LSMFM2)) that is specifically expressed in cardiomyocytes. As described herein, it was discovered that LSMEM2 is a specific in vitro biological marker of cardiomyocytes derived from human pluripotent stem cells and to human fetal and adult cardiomyocytes. Mouse anti-human monoclonal antibodies having high affinity for five different epitopes of the extracellular domain of LSMEM2 are useful for identifying and isolating highly homologous populations of human cardiomyocytes following differentiation of human pluripotent stem cells in vitro. It was further discovered that LSMEM2 is a specific biological marker of cardiomyocytes of both the atrium and ventricle of the adult human heart and, thus, is a useful marker of adult atrial and ventricular cardiomyocytes in vivo, ex vivo, and in situ. Accordingly, the present invention relates to biological markers and methods for identifying, generating, and purifying human cardiomyocytes.

Compositions of the Invention

Accordingly, provided herein are sensitive and specific biological markers of cardiomyocyte populations. Also provided herein are biological markers that are diagnostic or prognostic of certain cardiovascular injuries. As used herein, the terms “cardiomyocyte,” “cardiomyocyte cell,” and “cardiac myocytes” are used interchangeably and refer to primary cardiomyocytes, cardiomyocyte precursor cells, clonal cardiomyocytes derived from adult human heart, immortalized cardiomyocytes, human embryonic stem cell (hESC)-derived cardiomyocytes, human induced pluripotent stem cell (iPS)-derived cardiomyocytes, or any cell displaying cardiomyocyte-specific markers such that a pathologist, scientist, or laboratory technician would recognize the cell to be cardiomyocyte-specific or cardiomyocyte derived.

As used herein, the terms “biological marker” and “biomarker” are used interchangeably and refer to biological molecules (e.g., DNA, mRNA, protein) the expression of which is associated with cardiomyocytes at a given developmental, maturation, or differentiation stage. Biomarkers encompass, without limitation, genes, proteins, nucleic acids (e.g., circulating nucleic acids (CNA)) and metabolites, together with their polymorphisms, mutations, variants, modifications, subunits, fragments, protein-ligand complexes, and degradation products, protein-ligand complexes, elements, related metabolites, and other analytes or sample-derived measures. Biomarkers can also include mutated proteins or mutated nucleic acids. The skilled person will understand that instead of detecting the complete biomarker protein, one may also detect peptide fragments of said biomarker proteins, for example which are derived from the biomarker proteins by fragmentation thereof. The term peptide fragment as used herein refers to peptides having between 5 and 50 amino acids, for example 5, 10, 15, 20, 25, 30, 35, 40, 45 or 50 amino acids. These peptide fragments preferably provide a unique amino acid sequence of the protein, and are associated with the cardiovascular events as disclosed herein.

In one aspect, provided herein is a biological marker of cardiomyocytes derived from pluripotent stem cells. In particular, provided herein is a biological marker of cardiomyocytes derived from human embryonic stem cells (hESCs) or human induced pluripotent stem cells (iPSs). Upon in vitro differentiation of human pluripotent stem cells, cardiomyocytes can be identified from the result cell population on the basis of cell surface expression of LSMEM2. Since there were previously no specific cell surface markers for human pluripotent stem cell-derived cardiomyocytes or cardiomyocytes isolated or obtained by other methods, the monoclonal antibodies described herein provide for effective mono-specific probes which can be utilized for identifying, quantifying, and purifying cardiomyocytes from heterogeneous cell populations comprising multiple cardiomyocyte subtypes. Expression of LSMEM2 is also a useful biological marker of atrial and ventricular cardiomyocytes of the adult heart in vivo or in a biological sample (e.g., ex vivo, in situ). In such cases, ventricular and atrial cardiomyocytes of adult human heart are identified on the basis of cell surface expression of LSMEM2.

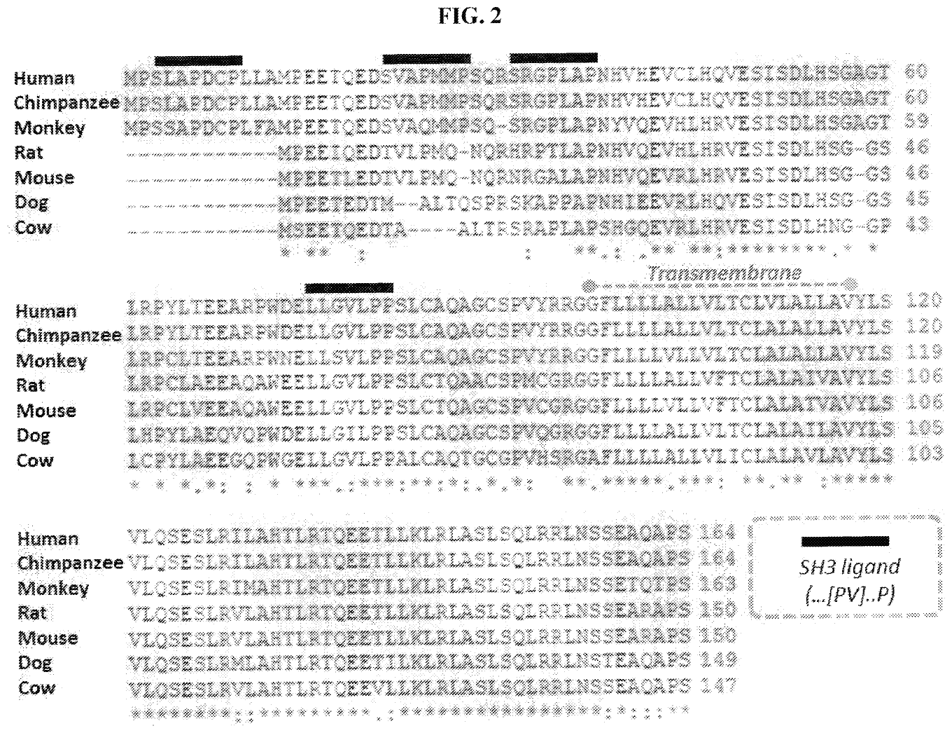

In some cases, a biomarker of the invention is a high affinity antibody having specificity for the cell surface protein LSMEM2 (SEQ ID NO:2) encoded by the nucleotide sequence SEQ ID NO:1 (GeneID 132228). For example, such a biomarker can be a monoclonal antibody (or antigen binding fragment thereof) that binds a polypeptide that comprises a sequence at least 80% homologous (and preferably 98% homologous) to the sequence from amino acid 1 to and including amino acid 164 of LSMEM2 (SEQ ID NO:2) (see FIG. 2).

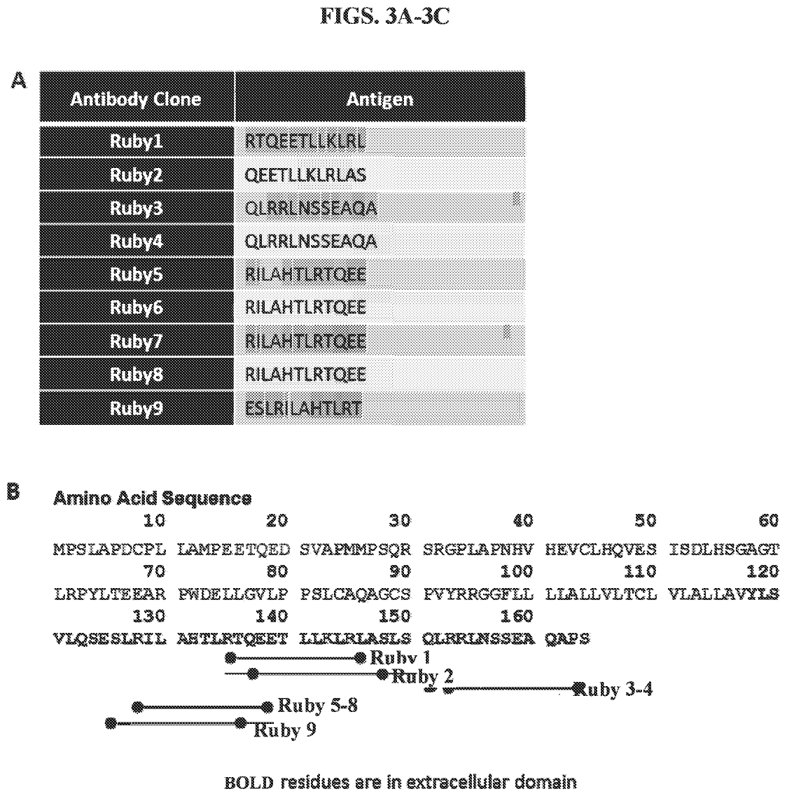

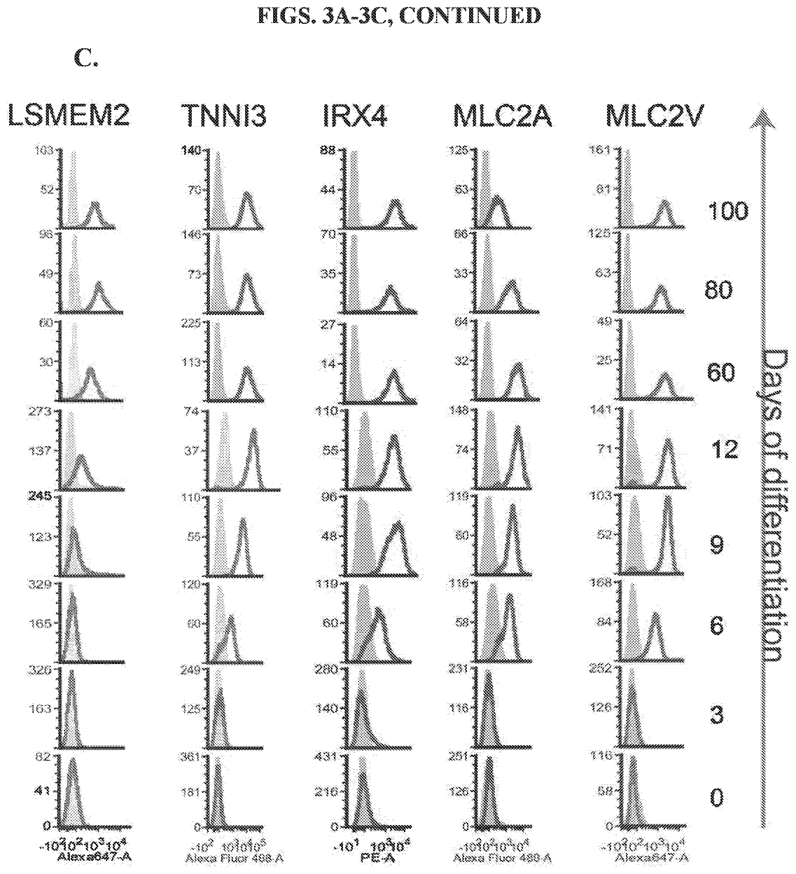

In exemplary embodiments, biomarkers provided herein are monoclonal antibodies and antibody fragments having specificity for an epitope of the cell surface protein LSMEM2. For example, FIG. 3 presents nine LSMEM2 monoclonal antibodies (Ruby1, Ruby2, Ruby3, Ruby4, Ruby5, Ruby6, Ruby7, Ruby8, and Ruby9) and the five LSMEM2 epitopes to which they bind (SEQ ID NOs:4-8). As used herein, the terms “epitope” and “antigenic determinant” refer to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996).

LSMEM2 biomarkers include whole antibodies as well as LSMEM2 antibody fragments having specificity for an epitope of LSMEM2. The terms “antibody fragment” and “antigen-binding fragment” refer to one or more fragments of an antibody that retain the ability to bind to an antigen. It has been shown that the antigen-binding function of an antibody can be performed by a portion of an intact antibody, generally the antigen binding or variable region. Examples of antibody fragments include Fab, Fab′, F(ab′)₂ and Fv fragments. Accordingly, LSMEM2 antibody fragments include, without limitation, Fab, Fab′, F(ab′)₂, Fv fragments, rIgG, diabodies, single chain antibodies, and multispecific antibodies having the desired specificity for an epitope of the cell surface protein LSMEM2. The term “monoclonal antibody” refers to an antibody that is derived from a single clone, including any eukaryotic, prokaryotic, or phage clone, and not the method by which it is produced. The term “monoclonal antibody” as used herein is not limited to antibodies produced through hybridoma technology. Monoclonal antibodies useful with the present invention may be prepared using a wide variety of techniques known in the art including the use of hybridoma, recombinant, and phage display technologies, or a combination thereof. For example, monoclonal antibodies can be prepared using hybridoma methods, such as those described by Kohler & Milstein, Nature 256:495 (1975); Harlow and Lane, “Antibodies: A Laboratory Manual,” Cold Spring Harbor Laboratory Press, New York (1988); Hammerling et al., in: “Monoclonal Antibodies and T-Cell Hybridomas,” Elsevier, N.Y. (1981), pp. 563-681 (all of which are incorporated herein by reference in their entireties). In a hybridoma method, a mouse, hamster, or other appropriate host animal, is typically immunized with an immunizing agent to elicit lymphocytes that produce or are capable of producing antibodies that will specifically bind to the immunizing agent. Alternatively, the lymphocytes may be immunized in vitro. The immunizing agent will typically include a polypeptide encoded by nucleic acid of SEQ ID NO:1 or a fragment thereof, or a fusion of protein sequence of SEQ ID NO:2 or fragments thereof (e.g., epitopes set forth as SEQ ID NOS:4-8).

In other cases, LSMEM2 biomarkers of the present invention are polyclonal antibodies. Methods of preparing polyclonal antibodies are known to the skilled artisan. For example, polyclonal antibodies can be raised in a mammal, e.g., by one or more injections of an immunizing agent and, if desired, an adjuvant. Typically, the immunizing agent and/or adjuvant will be injected in the mammal by multiple subcutaneous or intraperitoneal injections. The immunizing agent may include a protein encoded by a nucleic acid or fragment thereof or a fusion protein thereof. It may be useful to conjugate the immunizing agent to a protein known to be immunogenic in the mammal being immunized. Examples of such immunogenic proteins include, without limitation, serum albumin, bovine thyroglobulin, and soybean trypsin inhibitor. Examples of adjuvants which may be employed include Freund's complete adjuvant and MPL-TDM adjuvant (monophosphoryl Lipid A, synthetic trehalose dicorynomycolate). An immunization protocol may be selected by one skilled in the art without undue experimentation.

LSMEM2 biomarkers provided herein include chimeric, humanized, and human antibodies. As used herein, a “chimeric antibody” is an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. Methods for producing chimeric antibodies are known in the art. See e.g., Morrison, Science 229:1202-1207 (1985); Oi et al., BioTechniques 4:214-221 (1986); Gillies et al., J. Immunol. Methods 125:191-202 (1989); U.S. Pat. Nos. 5,807,715; 4,816,567; and 4,816,397, which are incorporated herein by reference in their entireties.

The term “humanized antibody” or “humanized immunoglobulin” refers to an immunoglobulin comprising a human framework, at least one and preferably all complementarity determining regions (CDRs) from a non-human antibody, and in which any constant region present is substantially identical to a human immunoglobulin constant region, i.e., at least about 85-90%, and preferably at least 95% identical. Hence, all parts of a humanized immunoglobulin, except possibly the CDRs, are substantially identical to corresponding parts of one or more native human immunoglobulin sequences. Accordingly, such humanized antibodies are chimeric antibodies (U.S. Pat. No. 4,816,567), wherein substantially less than an intact human variable domain has been substituted by the corresponding sequence from a non-human species. Often, framework residues in the human framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. See, e.g., Queen et al., U.S. Pat. Nos. 5,530,101; 5,585,089; 5,693,761; 5,693,762; 6,180,370 (each of which is incorporated by reference in its entirety). Antibodies can be humanized using a variety of techniques known in the art including, for example, CDR-grafting (EP 239,400; PCT publication WO 91/09967; U.S. Pat. Nos. 5,225,539; 5,530,101 and 5,585,089), veneering or resurfacing (EP 592,106; EP 519,596; Padlan, Mol. Immunol., 28:489-498 (1991); Studnicka et al., Prot. Eng. 7:805-814 (1994); Roguska et al., Proc. Natl. Acad. Sci. 91:969-973 (1994), and chain shuffling (U.S. Pat. No. 5,565,332).

Completely “human” antibodies may be desirable for therapeutic treatment of human patients. Human antibodies can be made by a variety of methods known in the art including phage display methods described above using antibody libraries derived from human immunoglobulin sequences. See U.S. Pat. Nos. 4,444,887 and 4,716,111; and PCT publications WO 98/46645; WO 98/50433; WO 98/24893; WO 98/16654; WO 96/34096; WO 96/33735; and WO 91/10741, each of which is incorporated herein by reference in its entirety. Human antibodies can also be produced using transgenic mice which are incapable of expressing functional endogenous immunoglobulins, but which can express human immunoglobulin genes. For an overview of this technology for producing human antibodies, see Lonberg and Huszar, Int. Rev. Immunol. 13:65-93 (1995). For a detailed discussion of this technology for producing human antibodies and human monoclonal antibodies and protocols for producing such antibodies, see, e.g., PCT publications WO 98/24893; WO 92/01047; WO 96/34096; WO 96/33735; European Patent No. 0 598 877; U.S. Pat. Nos. 5,413,923; 5,625,126; 5,633,425; 5,569,825; 5,661,016; 5,545,806; 5,814,318; 5,885,793; 5,916,771; and 5,939,598, which are incorporated by reference herein in their entireties.

LSMEM2 biological markers also encompass nucleic acids and polypeptide variants, alleles, mutants, and interspecies homologues that: (1) have a nucleotide sequence that has greater than about 60% nucleotide sequence identity, 65%, 70%, 75%, 80%, 85%, 90%, or more preferably 91%, 92%, 93%, 94%, 95%, 96%, 97%, 98% or 99% or greater nucleotide sequence identity, preferably over a region of at least about 25, 50, 100, 200, 500, 1000, or more nucleotides, to a nucleotide sequence of SEQ ID NO:1; (2) bind to antibodies, e.g., polyclonal antibodies, raised against an immunogen comprising an amino acid sequence encoded by SEQ ID NO:1, or a portion thereof, and conservatively modified variants thereof; (3) specifically hybridize under stringent hybridization conditions to at least a portion of the nucleic acid sequence, or the complement thereof, of SEQ ID NO:1 and conservatively modified variants thereof; or (4) have an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino sequence identity, preferably over a region of at least about 25, 50, 100, 200, or more amino acids, to an amino acid sequence of SEQ ID NO:2. A polynucleotide or polypeptide sequence is typically from a mammal including, but not limited to, primate, e.g., human; rodent, e.g., rat, mouse, hamster; cow, pig, horse, sheep, or other mammal. The terms “LSMEM2 polypeptide” and “LSMEM2 polynucleotide” include both naturally occurring or recombinant forms.

Also provided herein are host cells capable of producing LSMEM2 polypeptides or fragments thereof, including any of the LSMEM2 antibody embodiments. As used herein, the term “host cell” refers to a naturally occurring cell or a transformed cell that contains an expression vector and supports the replication or expression of the expression vector. Host cells may be cultured cells, explants, cells in vivo, and the like. Host cells may be prokaryotic cells such as E. coli, or eukaryotic cells such as yeast, insect, amphibian, or mammalian cells such as Chinese Hamster Ovary (CHO) cells, HeLa cells, and the like (see, e.g., the American Type Culture Collection catalog or web site atcc.org on the World Wide Web). Preferably, the host cell is selected from the group consisting of a CHO cell, E. coli, yeast cell, and insect cell.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer. As used herein, the term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an a carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

The terms “isolated,” “purified,” or “biologically pure” refer to material that is substantially or essentially free from components that normally accompany it as found in its native state. For example, the term “isolated” can refer to a cell or cell population that is not within its natural environment. In such cases, an isolated cell or cell population has been substantially separated from surrounding tissue or from the tissue, organ, or cell population from which they originated. Accordingly, the term “isolated” encompasses cells which have been removed from the organism from which they originated, and exist in culture. The term also encompasses cells which have been removed from the organism from which they originated, and subsequently re-inserted into an organism.

Purity and homogeneity are typically determined using analytical chemistry techniques such as polyacrylamide gel electrophoresis or high performance liquid chromatography. A protein, nucleic acid, or cell that is the predominant species present in a preparation is substantially purified. In particular, an isolated nucleic acid is separated from some open reading frames that naturally flank the gene and encode proteins other than protein encoded by the gene. The term “purified” in some embodiments denotes that a nucleic acid or protein gives rise to essentially one band in an electrophoretic gel. Preferably, it means that the protein, nucleic acid, or cell is at least 85% pure, more preferably at least 95% pure, and most preferably at least 99% pure. “Purify” or “purification” in other embodiments means removing at least one contaminant from the composition to be purified. In this sense, purification does not require that the purified compound be homogeneous, e.g., 100% pure. In some cases, a cell or cell population obtained according to a method provided herein is “substantially pure” or “substantially homogeneous.” As used herein, the terms “substantially pure” and “substantially homogeneous” refer to a population of cells that is at least about 75%, in some embodiments at least about 85%, in some embodiments at least about 90%, and in some embodiments at least about 95% pure, with respect to other cells that make up a total cell population. In other words, the terms “substantially pure” and “substantially homogeneous” refer to a population of cardiomyocytes of the present invention that contain fewer than about 25%, in some embodiments fewer than about 15%, in some embodiments fewer than about 5%, and in some embodiments less than 2% of non-cardiomyocyte cell types.

The methods of introducing exogenous nucleic acid into mammalian hosts, as well as other hosts, is well known in the art, and will vary with the host cell used. Techniques include dextran-mediated transfection, calcium phosphate precipitation, polybrene mediated transfection, protoplast fusion, electroporation, viral infection, encapsulation of the polynucleotide(s) in liposomes, and direct microinjection of the DNA into nuclei.

In some embodiments, LSMEM2 polypeptides and protein fragments are expressed in bacterial systems. Bacterial expression systems are well known in the art. Promoters from bacteriophage may also be used and are known in the art. In addition, synthetic promoters and hybrid promoters are also useful; e.g., the tac promoter is a hybrid of the trp and lac promoter sequences. Furthermore, a bacterial promoter can include naturally occurring promoters of non-bacterial origin that have the ability to bind bacterial RNA polymerase and initiate transcription. In addition to a functioning promoter sequence, an efficient ribosome binding site is desirable. The expression vector may also include a signal peptide sequence that provides for secretion of the LSMEM2 protein in bacteria. The protein is either secreted into the growth media (gram-positive bacteria) or into the periplasmic space, located between the inner and outer membrane of the cell (gram-negative bacteria). The bacterial expression vector may also include a selectable marker gene to allow for the selection of bacterial strains that have been transformed. Suitable selection genes include genes which render the bacteria resistant to drugs such as ampicillin, chloramphenicol, erythromycin, kanamycin, neomycin and tetracycline. Selectable markers also include biosynthetic genes, such as those in the histidine, tryptophan and leucine biosynthetic pathways. These components are assembled into expression vectors. Expression vectors for bacteria are well known in the art, and include vectors for Bacillus subtilis, E. coli, Streptococcus cremoris, and Streptococcus lividans, among others. The bacterial expression vectors are transformed into bacterial host cells using techniques well known in the art, such as calcium chloride treatment, electroporation, and others.

In one embodiment, LSMEM2 polypeptides are produced in insect cells. Expression vectors for the transformation of insect cells, and in particular, baculovirus-based expression vectors, are well known in the art.

LSMEM2 polypeptides also can be produced in yeast cells. Yeast expression systems are well known in the art, and include expression vectors for Saccharomyces cerevisiae, Candida albicans and C. maltosa, Hansenula polymorpha, Kluyveromyces fragilis and K. lactis, Pichia guillerimondii and P. pastoris, Schizosaccharomyces pombe, and Yarrowia lipolytica.

LSMEM2 polypeptides may also be made as a fusion protein, using techniques well known in the art. Thus, e.g., for the creation of monoclonal antibodies, if the desired epitope is small, the LSMEM2 protein may be fused to a carrier protein to form an immunogen. Alternatively, the LSMEM2 protein may be made as a fusion protein to increase expression, or for other reasons. For example, when the LSMEM2 protein is a LSMEM2 peptide, the nucleic acid encoding the peptide may be linked to other nucleic acid for expression purposes.

Human antibodies can be produced using various techniques known in the art, including phage display libraries (Hoogenboom and Winter, J. Mol. Biol. 227:381 (1991); Marks et al., J. Mol. Biol. 222:581 (1991)). Techniques are also available for the preparation of human monoclonal antibodies (Cole et al., Monoclonal Antibodies and Cancer Therapy, p. 77 (1985); Boerner et al., J. Immunol. 147(1):86-95 (1991)). Similarly, human antibodies can be made by introducing of human immunoglobulin loci into transgenic animals, e.g., mice in which the endogenous immunoglobulin genes have been partially or completely inactivated. For example, one can engineer a mouse strain deficient in mouse antibody production using large fragments of the human Ig loci such that the mice produce human antibodies in the absence of mouse antibodies. Large human Ig fragments may preserve the large variable gene diversity as well as the proper regulation of antibody production and expression. Upon challenge, human antibody production is observed, which closely resembles that seen in humans in all respects, including gene rearrangement, assembly, and antibody repertoire. This approach is described, e.g., in U.S. Pat. Nos. 5,545,807; 5,545,806; 5,569,825; 5,625,126; 5,633,425; 5,661,016, and in the following scientific publications: Marks et al., Bio/Technology 10:779-783 (1992); Lonberg et al., Nature 368:856-859 (1994); Morrison, Nature 368:812-13 (1994); Fishwild et al., Nature Biotechnology 14:845-51 (1996); Neuberger, Nature Biotechnology 14:826 (1996); Lonberg & Huszar, Intern. Rev. Immunol. 13:65-93 (1995). By exploiting the mouse machinery for antibody diversification and selection and the lack of immunological tolerance to human proteins, the reproduced human antibody repertoire in these mouse strains may yield high affinity antibodies against any antigen of interest, including human antigens. Using hybridoma technology, antigen-specific human mAbs with the desired specificity may be produced and selected.

In some cases, LSMEM2 biomarkers provided herein are bispecific antibodies. As used herein, the terms “bispecific antibody” and “bifunctional antibody” refer to monoclonal, preferably human or humanized, antibodies that have binding specificities for at least two different antigens or that have binding specificities for two epitopes on the same antigen. Bispecific or bifunctional antibodies are typically artificial hybrid antibodies having two different heavy/light chain pairs and two different binding sites. Bispecific antibodies may be produced by a variety of methods including, without limitation, fusion of hybridomas or linking of Fab′ fragments. See, e.g., Songsivilai and Lachmann, Clin. Exp. Immunol. 79: 315-321 (1990), Kostelny et al., J. Immunol. 148:1547-1553 (1992).

In some cases, LSMEM2 biomarkers provided herein are labeled with suitable radioactive, enzymatic, or fluorescent labels by conventional methods and/or bound to suitable solid carriers, which will be apparent to those skilled in the art. For example, the monoclonal antibodies can be used in combination with, or coupled to, an immunochemical such as fluorescein isothiocyanate, peroxidase, biotin and its analogs (e.g., iminobiotin), avidin and its analogs (streptavidin), alkaline phosphatases, or other such markers. In particular embodiments, LSMEM2 biomarkers can be bound or attached to certain substrates and utilized to capture ventricular cardiomyocytes when heterogeneous stem cell-derived cell populations are brought in contact with the attached monoclonal antibodies. The bound cells may then be separated from the solid phase by known methods depending essentially upon the nature of the solid phase and the antibody. The unbound cells can be recovered and used for various therapeutic purposes such as for the regeneration of bone, etc., depending upon the various external and internal factors.

In another aspect, provided herein are circulating biomarkers for conditions associated with cardiovascular injury. Cell surface proteins are often shed and appear as informative circulating biomarkers in numerous diseases including cancer and heart disease (Palmer et al., (2008) PLoS One 3, e2633; Wojtalewicz et al. (2014) PLoS One 9, e90461; Eleuteri et al., (2014) Biomarkers 19, 214-22). Cell surface proteins can also be present in microvesicles or exosomes shed from the plasma membrane of cells and subsequently detected in bodily fluids including blood, serum, plasma, or urine (Boukouris et al., (2015) Proteomics Clin Appl, 9, 358-67; Lasser, (2015) Expert Opin Biol Ther, 15, 103-17). This class of biomarker can be especially informative in cases where the protein is unique to a single cell type in the body. For example, a portion of LSMEM2 that is shed from the surface of a cardiomyocyte may be detectable in the circulation or urine as an accessible biomarker. Such detection could be performed by antibody based methods or by mass spectrometry based methods, or a combination of both. The extracellular domain of LSMEM2 is cleaved from the cell surface of iCM in vitro and is detectable in the cell culture supernatant using the anti-LSMEM2 monoclonal antibodies provided herein.

Methods of the Invention

In another aspect, provided herein are methods for detecting LSMEM2-encoding polynucleotide sequences, LSMEM2 polypeptides, and LSMEM2-expressing cells using biomarkers provided herein and/or any of a number of well recognized detection assays. Standard methods include, for example, radioisotope immunoassay, enzyme-linked immunosorbent assay (ELISA) (Engvall et al., Methods in Enzymol. 70:419-439 (1980)), SISCAPA (Stable Isotope Standards and Capture by Anti-Peptide Antibodies) (Anderson et al., J Proteome Res. 2004 Mar.-Apr.; 3(2):235-44), mass spectrometry, immunofluorescence assays, Western blot, affinity chromatography (affinity ligand bound to a solid phase), fluorescent antibody assays, immunochromatography, and in situ detection with labeled antibodies. See, e.g., U.S. Pat. Nos. 4,366,241; 4,376,110; 4,517,288; and 4,837,168). Although any appropriate method can be selected, taking various factors into consideration, ELISA methods are particularly sensitive.

In some cases, a method of detecting LSMEM2-expressing cells comprises flow cytometry, fluorescence activated cell sorting (FACS), magnetic assisted cell sorting (MACS), or another cell analysis or cell sorting method. Flow cytometry is a powerful analytical tool for assessing the quality of cells in culture and determining subpopulation homogeneity, and with proper experimental design, can provide quantitative measurements. For quantitative measurements using flow cytometry, monoclonal antibodies are particularly advantageous. See, e.g., Bhattacharya et al., Journal of Visualized Experiments 2014, 91(e52010).

In another aspect, the present invention provides methods for isolating and purifying LSMEM2-expressing cells. In particular, the present invention provides non-transgenic, scalable methods that employ monoclonal antibodies provided herein to separate pluripotent stem cell-derived cardiomyocytes from other stem cell-derived cell types such as nodal and atrial cardiomyocytes. For example, a further embodiment of the present invention is directed to a method of producing a population of pluripotent stem cell-derived cardiomyocytes. The method comprises or consists essentially of providing a population of stem cell-derived cardiomyocytes, where the population comprises cardiomyocytes; contacting the cell suspension with monoclonal antibodies which recognize an epitope of LSMEM2 present on the cell surface of cardiomyocytes but do not recognize an epitope on non-cardiomyocytes; and separating and recovering from the contacted cell suspension the cardiomyocytes bound by the monoclonal antibodies. Such methods can be used to obtain a sufficient quantity of cardiomyocytes for transplantation for the treatment of myocardial infarction. Advantageously, the methods provided herein yield populations of cardiomyocytes that are substantially free of non-cardiomyocyte cell types.

Preferably, a method for isolating a population of in vitro differentiated cardiomyocytes comprises the steps of providing a cell suspension comprising human pluripotent stem cell-derived cardiomyocytes; contacting the cell suspension with one or more biomarkers provided herein (e.g., a monoclonal antibody for an epitope of LSMEM2); detecting binding of the biomarker to cardiomyocytes but not on other cell types; and recovering the biomarker-bound cells.

Preferably, an isolated population of in vitro differentiated cardiomyocytes essentially comprises only cells of the invention, i.e., the cell population is pure. In many aspects, the cell population comprises at least about 80% (in other aspects at least 85%, 90%, 91%, 92%, 93%, 94%, 95%, 96%, 97%, 98%, 99%, 99.5%, 99.9% or 100%) of the ventricular cardiomyocytes of the invention.

In some cases, a cell population obtained according to the methods provided herein is characterized by a distinctive expression profile for certain markers. Expression of markers associated with cardiac commitment and stage- and chamber-specificity may be detected through the use of an RT-PCR experiment (e.g., qRT-PCR) or through fluorescence activated cell sorting (FACS). For example, isolated cells can be assayed for expression of HCN4 (pacemaker marker, potassium/sodium hyperpolarization-activated cyclic nucleotide-gated channel 4), MLC1v (ventricle-specific intracellular marker), KCNA5 (potassium channel, voltage-gated, shaker-related subfamily A, member 5), MLC1a (atrial marker), MYH11 (smooth muscle marker), and MYOD1 (skeletal muscle marker). Markers of cardiomyocyte identity include TNNI3. Markers of ventricular cardiomyocyte subtype identity include IRX4 and MLC2v, and atrial cardiomyocyte subtype identity include COUP-TFII and MLC1v. At the protein level, IRX4 has been shown to be restricted to the ventricle from linear heart tube through neonatal stages in the mouse (Nelson et al., Dev Dyn. 2013, 243:381-392). It should be appreciated that this list is provided by way of example only, and is not intended to be limiting.

In some cases, biomarkers provided herein can be used in combination with other cardiomyocyte markers (e.g., a marker panel). For example, cardiomyocytes can be identified and isolated using a biomarker panel comprising LSMEM2 biomarkers provided herein in combination with other markers associated with cardiac commitment. In other cases, action potential measurements through electrophysiological or optical imaging experiments can be used to definitively assign cardiomyocyte type identity with regards to atrial, ventricular, and nodal.

An isolated cell population of the invention is considered to express a marker if at least about 70% of the cells of the population show detectable expression of the marker. In other aspects, at least about 80%, at least about 90% or at least about 95% or at least about 97% or at least about 98% or more of the cells of the population show detectable expression of the marker. In certain aspects, at least about 99% or 100% of the cells of the population show detectable expression of the markers.

In a further aspect, provided herein are methods for detecting a cardiovascular injury. In particular, provided herein are methods comprising detecting particular biomarkers to predict, monitor, and diagnosis a condition associated with cardiovascular injury. In some cases, detecting a biomarker comprises determining mRNA levels thereof in a subject's biological sample (e.g., blood). Detection of cardiomyocyte biomarkers as provided herein is useful for diagnosing a cardiovascular injury and determining the degree of severity of injury, the cell(s) involved in the injury, and/or the localization of the injury. As described in Example 4, mRNA levels of LSMEM2 are decreased in human failing heart as compared to non-failing control. As used herein, the term “injury” or “cardiovascular injury” is intended to include any damage or structural change that directly or indirectly affects the normal functioning of the cardiovascular system. By way of non-limiting example, the injury can be damage to the heart due to heart failure, myocardial infarction (including non-ST segment elevation myocardial infarction (NSTEMI) and ST segment elevation myocardial infarction (STEMI)), acute coronary syndrome, stable ischemic heart disease, unstable ischemic heart disease, ischemic cardiomyopathy, cardiac remodeling, or cardiac dilation. Cardiac remodeling a physiologic and pathologic condition that may occur after myocardial infarction (MI), pressure overload (aortic stenosis, hypertension), inflammatory heart muscle disease (myocarditis), idiopathic dilated cardiomyopathy, or volume overload (valvular regurgitation), and that manifests clinically as morphological changes in size, shape, and function of a cardiac tissue. Examples of cardiac remodeling include increase in cardiac hypertrophy and a sustained increase in cardiac chamber dimensions—i.e., pathological cardiac dilation—associated with an increase in the unstressed cardiac volume.

In exemplary embodiments, methods for detecting a cardiovascular injury comprise detecting expression of a biomarker and/or detecting differential expression and/or characteristic (e.g. post-translational modification, truncation, cleavage) of the biomarker between two or more samples (e.g., a test sample and a control sample). A biomarker is differentially present between the two samples if the amount of the biomarker in one sample is statistically significantly different from the amount of the biomarker in the other sample. As used herein, the phrase “differentially expressed” refers to differences in the quantity and/or the frequency of a biomarker present in a sample taken from patients having, for example, a cardiac injury as compared to a control subject.

For example, without limitation, a biomarker can be a polypeptide which is present at an elevated level or at a decreased level in samples of patients with myocardial injury as compared to samples of control subjects. A biomarker can be differentially present in terms of quantity, frequency or both. In some cases, a biomarker is differentially present between the two samples if it is present at least about 120%, at least about 130%, at least about 150%, at least about 180%, at least about 200%, at least about 300%, at least about 500%, at least about 700%, at least about 900%, or at least about 1000% greater than it is present in the other sample, or if it is detectable in one sample and not detectable in the other. Differences also include qualitative characteristics that may differ among samples and control subjects, such as amino acid residues that are post-translationally modified, differences in stoichiometry of multiple post-translational modifications across the protein, differences in cleavage, truncation, or degradation of the protein, or any modification that affects tertiary structure. These differences may be in combination with, or independent, of changes in quantity.

Alternatively (or additionally), a biomarker is differentially present between the two sets of samples if the frequency of detecting the biomarker in samples of patients suffering from for example, cardiovascular injury, is statistically significantly higher or lower than in the control samples. For example, a biomarker is differentially present between the two sets of samples if it is detected at least about 120%, at least about 130%, at least about 150%, at least about 180%, at least about 200%, at least about 300%, at least about 500%, at least about 700%, at least about 900%, or at least about 1000% more frequently or less frequently observed in one set of samples than the other set of samples.

As used herein, the terms “detect” and “detection” refer to identifying the presence, absence, or amount of the object to be detected. A “biological sample” or “sample” in the context of the methods provided herein is a biological sample isolated from a subject and can include, by way of non-limiting example, whole blood, blood fraction, serum, plasma, cerebrospinal fluid (CSF), urine, saliva, sputum, ductal fluid, bronchioaveolar lavage, blood cells, tissue biopsies, a cellular extract, a muscle or tissue sample, a muscle or tissue biopsy, or any other secretion, excretion, or other bodily fluids, including proximal fluids such synovial fluid, ductal lavage, and tissue interstitial fluid. Samples can be taken from a subject at defined time intervals (e.g., hourly, daily, weekly, or monthly) or at any suitable time interval as would be performed by those skilled in the art.

In some cases, the biomarkers provided herein are cell-type specific surface proteins useful as informative circulating biomarkers that can be detected independently of cell death (i.e., necrosis or apoptosis not required for biomarker release) using non-invasive methods.

The actual measurement of levels of a biomarker provided herein can be determined at the protein or nucleic acid level using any method(s) known in the art. A molecule or analyte such as a protein, polypeptide or peptide, or a group of two or more molecules or analytes such as two or more proteins, polypeptides or peptides, is “measured” in a sample when the presence or absence and/or quantity of said molecule or analyte or of said group of molecules or analytes is detected or determined in the sample, preferably substantially to the exclusion of other molecules and analytes. The terms “quantity”, “amount” and “level” are synonymous and generally well-understood in the art. The terms as used herein may particularly refer to an absolute quantification of a molecule or an analyte in a sample, or to a relative quantification of a molecule or analyte in a sample, i.e., relative to another value such as relative to a reference value as taught herein, or to a range of values indicating a base-line expression of the biomarker. These values or ranges can be obtained from a single patient or from a group of patients.

An absolute quantity of a molecule or analyte in a sample may be advantageously expressed as weight or as molar amount, or more commonly as a concentration, e.g., weight per volume or mol per volume.

The biomarkers disclosed herein can also be used to generate a “subject biomarker profile” taken from subjects who have cardiovascular injury. The subject biomarker profiles can be compared to a reference biomarker profile to diagnose or identify subjects at risk for developing cardiovascular injury, to monitor the progression of disease, as well as the rate of progression of disease, and to monitor the effectiveness of cardiovascular injury treatment modalities or subject management.

The term “subject” or “patient” as used herein typically denotes humans, but may also encompass reference to non-human animals, preferably warm-blooded animals, more preferably mammals, such as, e.g., non-human primates, rodents, canines, felines, equines, ovines, porcines, and the like.

By “assessing susceptibility” to a condition, it is meant that the method may comprise detecting the condition, diagnosing the condition, determining the severity of the condition, monitoring progress of the condition, determining a status of the condition or predicting the condition. Thus the present methods may be used, for example, to predict the likelihood of the condition developing in an individual at a future time as well as to detect the current presence of the condition. In specific embodiments, the methods may provide quantitative results which allow the progress of the condition to be continuously monitored, for instance to determine whether the condition is in an early or late stage of development or whether the individual is mildly, moderately or severely affected.

The terms “diagnosing” or “diagnosis” generally refer to the process or act of recognizing, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition).

In a further aspect, provided herein is a method for developing therapeutic agents for treating or preventing heart failure. For example, activators and inhibitors can be obtained for protease enzymes that specifically cleave LSMEM2, where cleavage of LSMEM2 is associated with onset, progression, or inhibition of cardiovascular disease. In another embodiment, inhibitors or activators can be made against LSMEM2 itself.

In another aspect, provided herein is a method of using a biomarker provided herein to quantify myocardial recovery in a subject having advanced heart failure. Of the 5.7 million Americans with heart failure, ˜10% will fail to respond to medical therapy and progressively worsen to develop advanced heart failure, for which the only definitive therapy is cardiac transplantation. As the supply of suitable donor hearts is limited to approximately 2000 per year in the United States, the care of advanced heart failure patients requires therapeutic alternatives. For some patients, mechanical circulatory support in the form of a ventricular assist device (VAD)—an implantable pump—can provide short-term or long-term support and in 1-2% of VAD recipients, the heart improves to the point where the pump can be removed, termed myocardial recovery. Currently, it is not possible to predict which heart failure patients will respond to medical therapy alone, which will benefit from VAD support, and which have no potential for recovery. For recipients of VAD therapy, standard methods for quantifying myocardial recovery to inform, for example, if and when to explant the device, require invasive testing. See, e.g., Mahr and Gundry, Proteomics Clin Appl. 2015; 9(0):121-133. For example, the process of clinically quantifying myocardial recovery is not yet well-defined, but often includes cardiac catheterization, echocardiography, and metabolic stress testing (Mann et al., Journal of the American College of Cardiology 2012; 60:2465-2472). Accordingly, provided herein is a method comprising quantifying LSMEM2-expressing cells using a LSMEM2-specific biomarker of the invention to quantitatively measure cardiac recovery. The method can comprise measuring the level of expression of LSMEM2 in the subject or in a biological sample obtained from a subject; and comparing the level of LSMEM2 with the LSMEM2 level from a control sample, wherein a measured level that is different than the control level or characteristic is indicative of a myocardial recovery. In some cases, the method is performed using a real-time imaging technique (e.g., immuno-PET) or using an ex-vivo strategy (e.g., live cell flow cytometry or immunofluorescence imaging of heart tissue) to track, for example, myocardial mass and structure.

Articles of Manufacture

In a further aspect, provided herein are kits comprising any or all of the following: assay reagents, buffers, and a LSMEM2-specific biomarker of the invention (e.g., an antibody, an oligonucleotide sequence complementary to a portion of a biomarker).

In some cases, provided herein is an enzyme-linked immunosorbent assay (ELISA) kit for detecting LSMEM2 polypeptide, where the kit comprises a monoclonal antibody provided herein and, in some cases, an enzyme-labeled reagent. In such cases, the labeling enzyme of the enzyme labeled reagent of the kit is horseradish peroxidase or alkaline phosphatase. In other cases, the monoclonal antibody is conjugated to a label such as a detectable marker, a cytotoxic agent, or an antibiotic. The detectable marker can be a radioisotope or an enzyme. The kit can further comprise a LSMEM2 standard solution, a color developing solution, and a reaction stopping solution. Kits provided herein can be used to rapidly detect LSMEM2 in a sample (e.g., a population of pluripotent stem cell-derived cardiomyocytes).

Also provided herein are kits that are useful in detecting a cardiovascular injury or event in an individual, wherein the kit can be used to detect a cardiovascular injury biomarker described herein. Preferably, the kits of the present invention comprise at least one cardiovascular injury-specific biomarker (e.g., an antibody, an oligonucleotide sequence complementary to a portion of the biomarker nucleic acid) that can be used to generate biomarker profiles according as set forth herein. The biomarker(s) may be part of an array, or the biomarker(s) may be packaged separately and/or individually. The kit may also comprise at least one internal standard to be used in generating the biomarker profiles of the present invention. Likewise, the internal standards can be any of the classes of compounds described above. The kits of the present invention also may contain reagents that can be used to detectably label biomarkers contained in the biological samples from which the biomarker profiles are generated. For this purpose, the kit may comprise a set of antibodies or functional fragments thereof that specifically bind one or more epitopes of a LSMEM2 polypeptide. In some cases, antibodies or functional fragments thereof provided in the kit are detectably labeled.

Preferably, kits further include instructional materials containing directions (i.e., protocols) for the practice of the methods of this invention. For example, the kits can include instructional materials for use of the kit to detect a cardiovascular injury biomarker described herein, which is differentially present in samples of cardiovascular injury subjects and normal subjects. The kits of the invention have many applications. For example, the kits can be used in any one of the methods of the invention described herein, such as, inter alia, to differentiate if a subject has cardiovascular injury, thus aiding a diagnosis. In another example, the kits can be used to identify compounds that modulate expression of one or more of the biomarkers in in vitro or in vivo animal models.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

The invention will be more fully understood upon consideration of the following non-limiting Examples.

### EXAMPLES

### Example 1—LSMEM2 mRNA During Differentiation and in Human Tissues

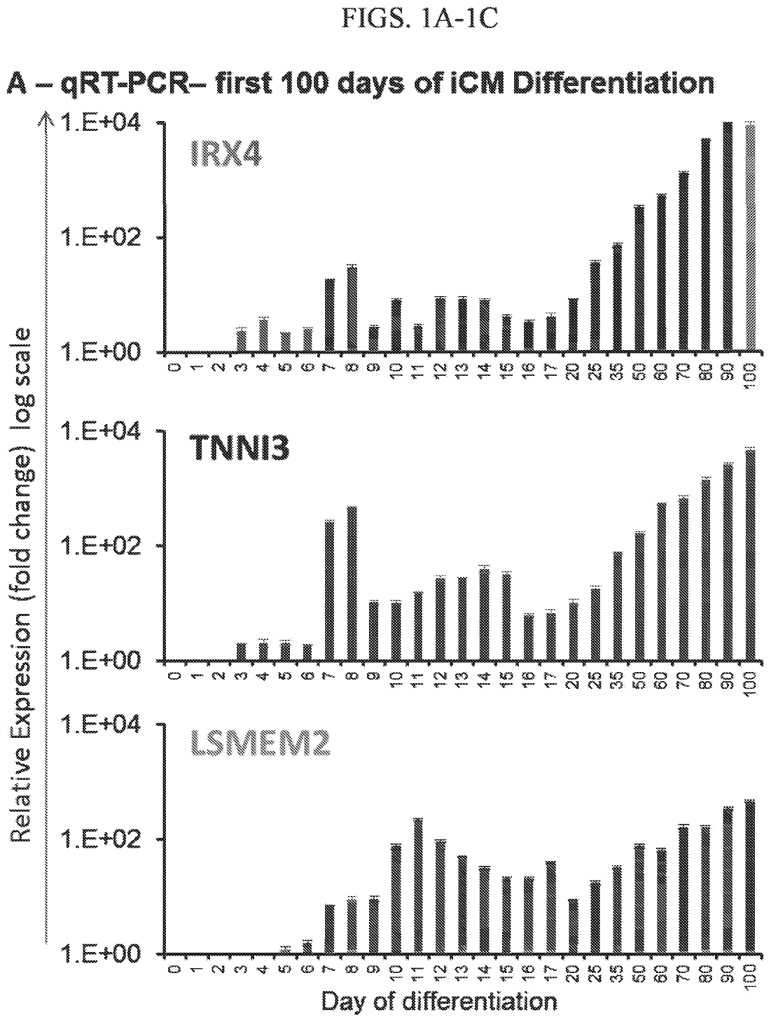

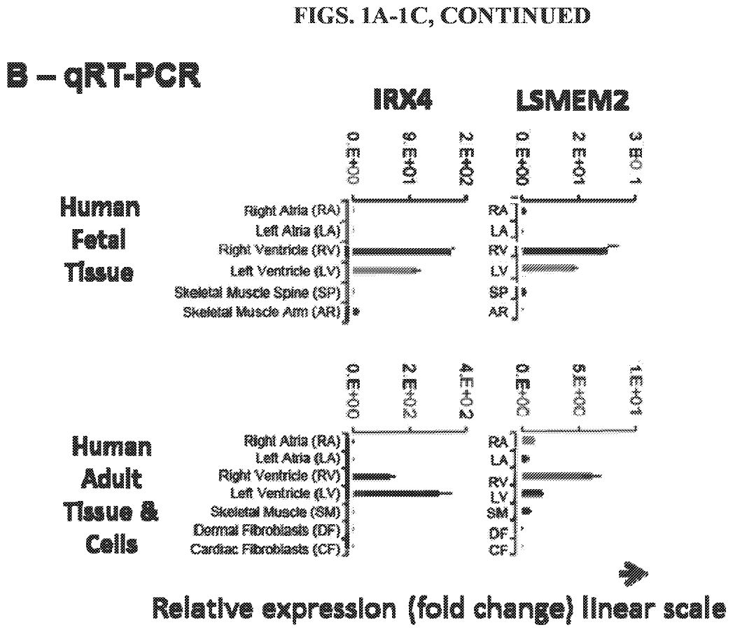

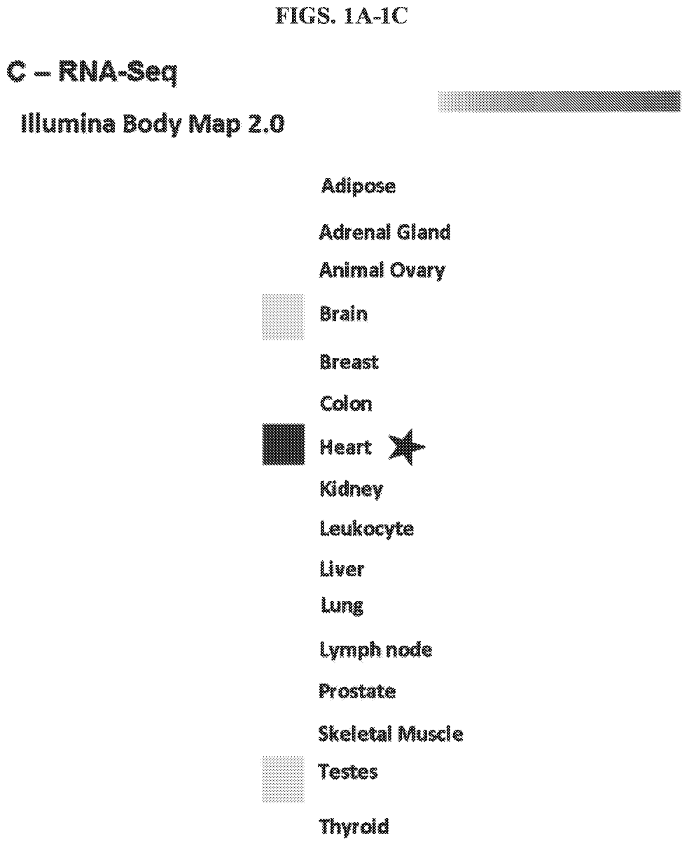

Human pluripotent stem cell (hPSC) models of induced cardiomyocyte (iCM) differentiation recapitulate the sequence of cardiac development, culminating in spontaneous and rhythmic contractions of iCM that mimic cells at embryonic/fetal stages of heart development in terms of electrophysiological signals, ion channel and gene expression patterns. See, e.g., Davis et al., Trends Mol Med. 17(9):475-84 (2011); Burridge et al., Cell Stem Cell 10(1):16-28 (2012); Cao et al., PLoS One 3(10):e3474 (2008); Mummery et al., Circulation 107(21):2733-40 (2003); Beqqali et al., Stem Cells 24(8):1956-67 (2006). Leucine-rich single-pass membrane protein 2 (LSMEM2) had not been previously reported as a cardiomyocyte marker, but our analyses reveal it is robustly expressed specifically in the human fetal and adult heart. During in vitro iCM differentiation, LSMEM2 mRNA is detected immediately after expression of TNNI3 and IRX4 (intracellular markers of cardiomyogenic and ventricular identity, respectively) is first detected (FIG. 1A). Quantitative RT-PCR (qRT-PCR) analyses of human tissues revealed that LSMEM2 was undetectable in fetal brain, kidney, liver, and spleen (data not shown) but was robustly expressed in the heart. In human fetal and adult heart, LSMEM2 was robustly expressed in the ventricles (FIG. 1B), with mRNA levels in adult atria and skeletal muscle near threshold of detection by qRT-PCR. RNA-Seq analysis of adult tissues showed restriction of LSMEM2 transcripts to the heart (FIG. 1C).

The Rat Genome Database (available at rgd.mcw.edu on the World Wide Web) was used to assess quantitative trait loci to reveal genomic locations where a genetic marker is linked to a phenotype. This analysis revealed a number of LSMEM2 loci related to cardiovascular pathology, including hypertension and cardiac mass. Moreover, two non-synonymous strain-specific sequence variants in exons are observed in three separate rat hypertension models. Also, as 511 bp of the LSMEM2 gene were antisense to Interferon-related developmental regulator 2, which is predominantly expressed in skeletal and cardiac muscle during early embryogenesis (Safran et al., Bioinformatics. 2002; 18(11):1542-3), our analysis raised the possibility of regulated alternate expression. Two separate studies showed that cardiomyocytes from patients having dilated cardiomyopathy exhibit reduced transcriptional levels of LSMEM2 (Sun et al., Sci Transl Med. 2012; 4(130):130ra47; Wittchen et al., J Mol Med (Berl). 2007; 85(3):257-71).

### Example 2—Monoclonal Antibodies to LSMEM2

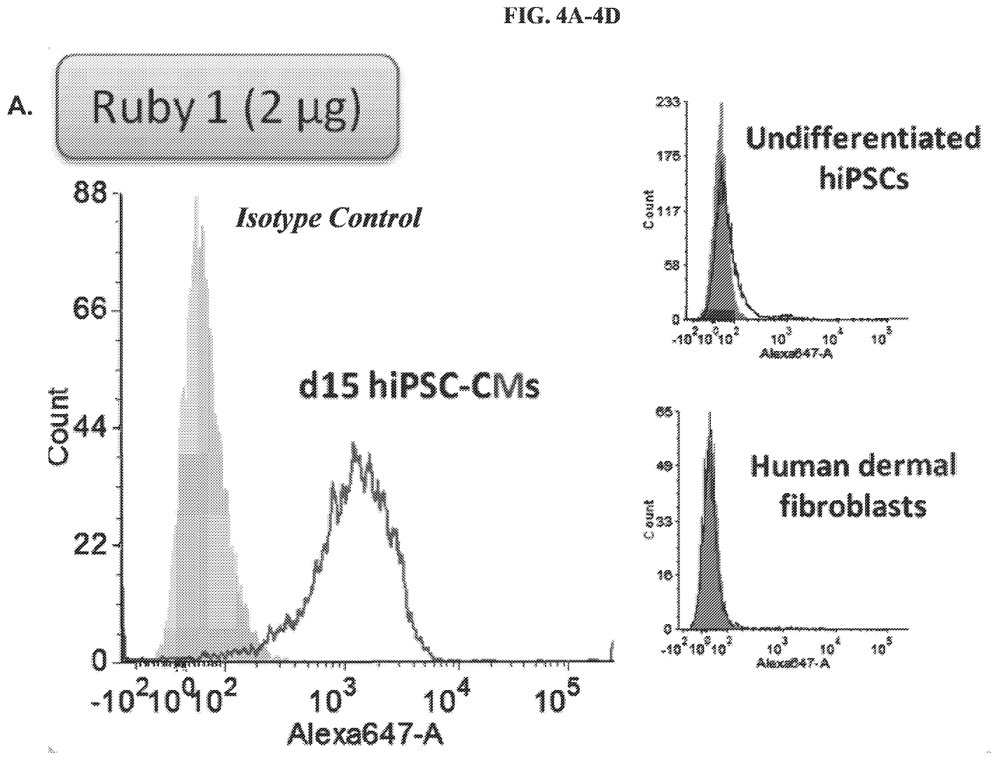

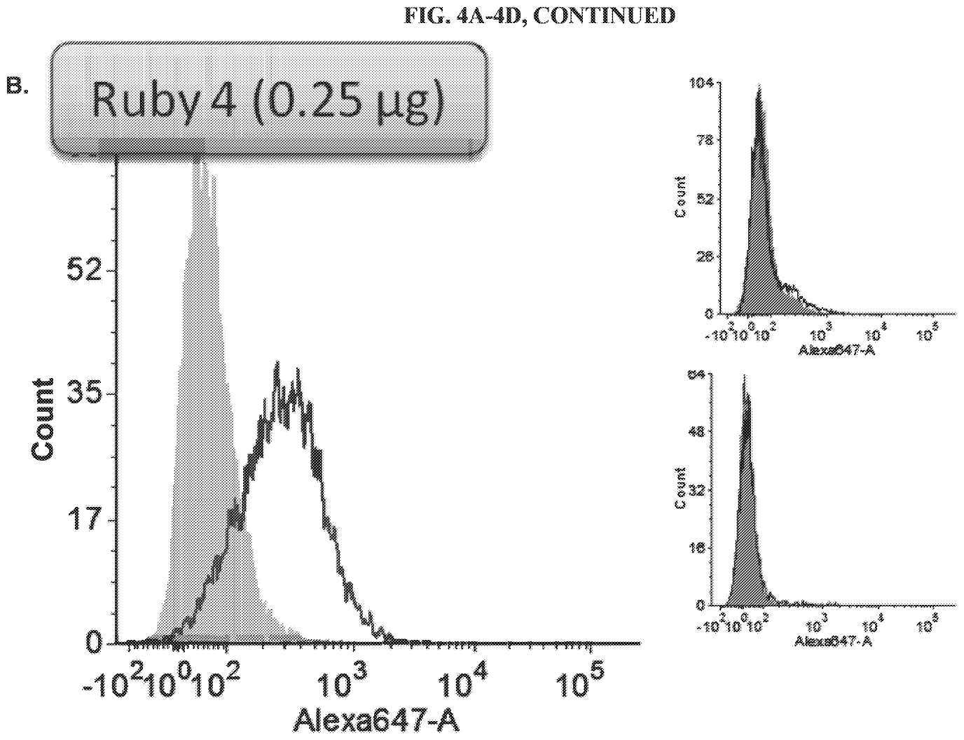

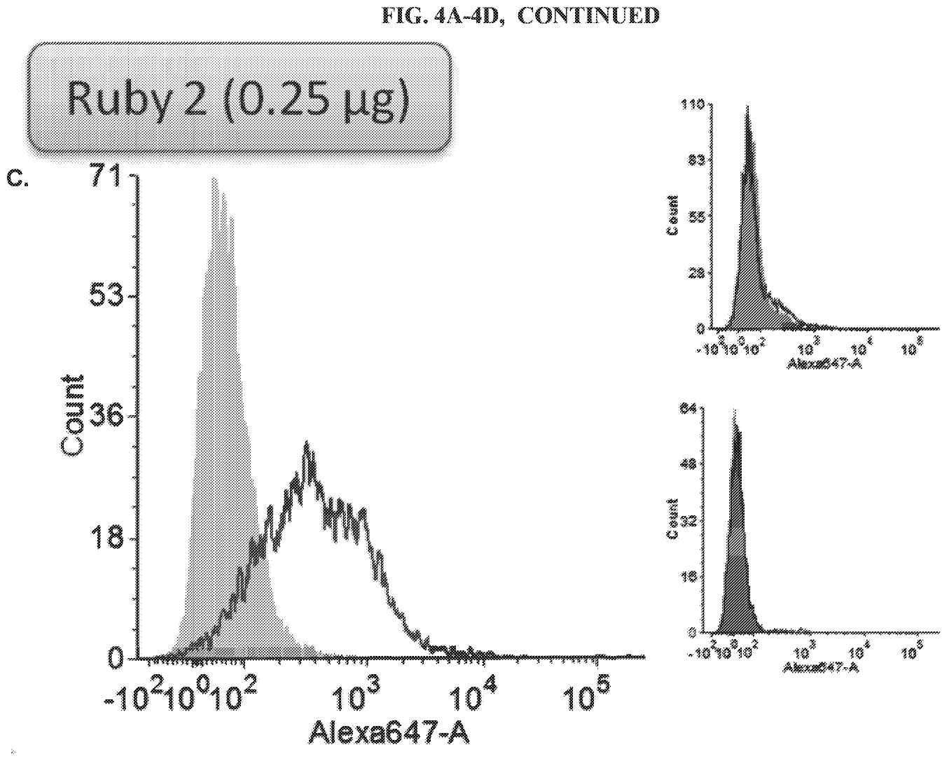

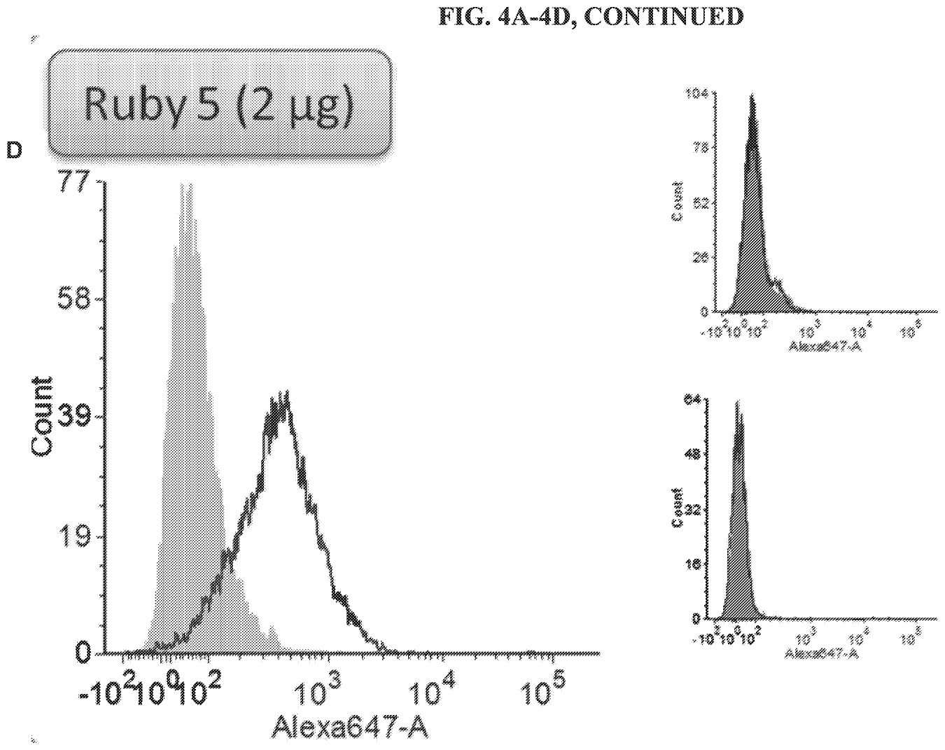

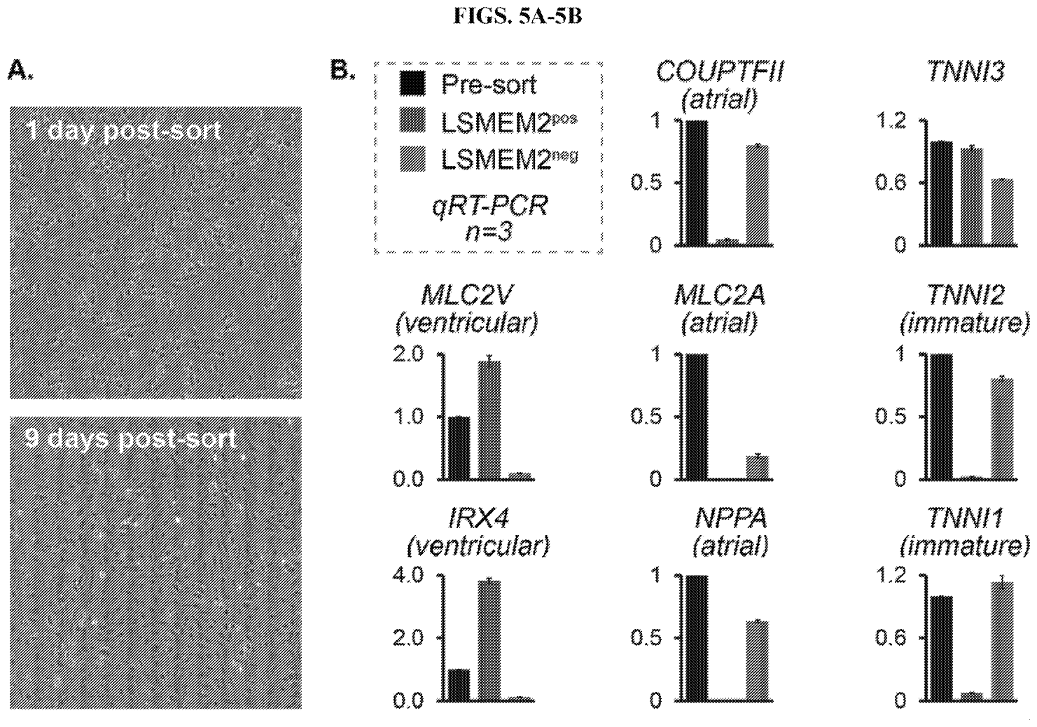

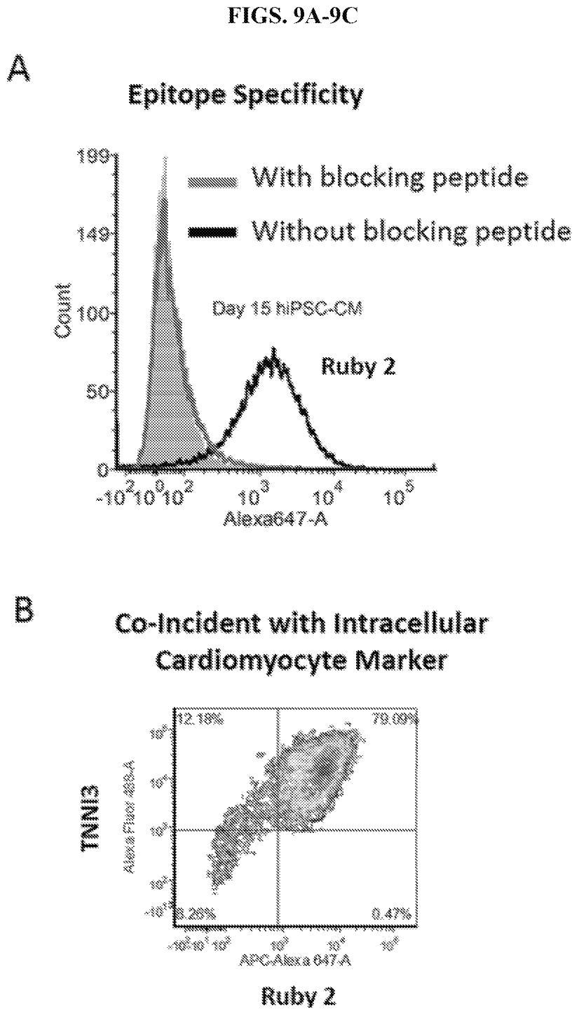

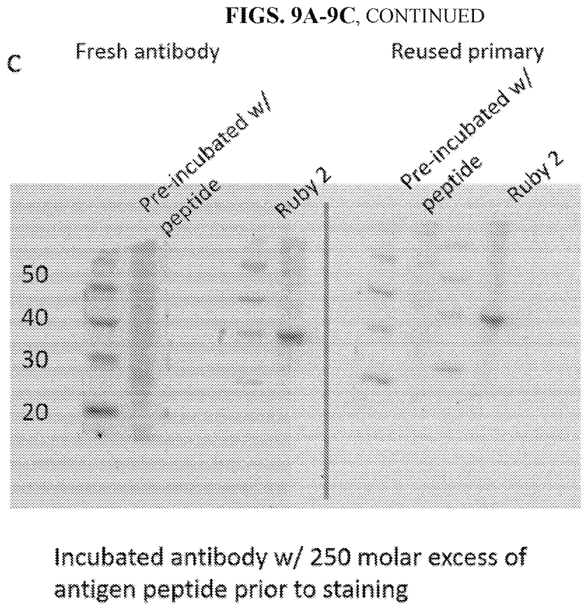

Prior to our proteomic analyses, LSMEM2 expression was previously documented only at the transcript level, with no known information regarding cellular sub-localization or transmembrane orientation. The extracellular domain of LSMEM2 was confirmed using the CSC-Technology data (including N-glycosylation site), which was then used in the epitope design process (FIG. 3A). We generated five human LSMEM2 monoclonal antibodies against four distinct extracellular epitopes and demonstrated that the mAbs successfully recognized native LSMEM2, as evidenced by live cell flow cytometry (data for four mAbs are presented in FIG. 4). Blocking with purified peptide antigen eliminates mAb binding in flow cytometry (FIG. 9A), immunostaining (not shown), and Western blotting (FIG. 9C), confirming their specificity. Importantly, LSMEM2 is absent on undifferentiated hiPSCs and cardiac and dermal fibroblasts (FIG. 4). Using mAb #1 (Ruby 1 in FIG. 3) to profile surface abundance during iCM differentiation, LSMEM2 appears on the surface by day 12 and persists beyond day 100 (FIG. 3C). Consistent with mRNA in FIG. 1A, LSMEM2 protein appears after TNNI3 and IRX4 (FIG. 3C). To determine whether LSMEM2 mAbs could be valuable for selecting iCM subtypes, day 10 iCM (a time point at which cells are committed cardiomyocytes, but are of mixed subtypes) were sorted via fluorescence activated cell sorting (FACS). Sorted cells are viable (FIG. 5A) and mRNA of reference markers show LSMEM2⁺ cardiomyocytes are enriched for ventricular markers and depleted of atrial markers and early troponin isoforms (FIG. 5B). Altogether, these data demonstrate that mAbs to LSMEM2 can identify and isolate cardiomyocytes from a mixed cell population. In a co-labeling experiment assessed by flow cytometry, the presence of Ruby 2 is co-incident with TNNI3, indicating that Ruby 2 is present on the cell surface of cardiomyocytes (FIG. 9B).

### Example 3—Monoclonal Antibodies for Analyzing Human and Mouse Heart Tissue

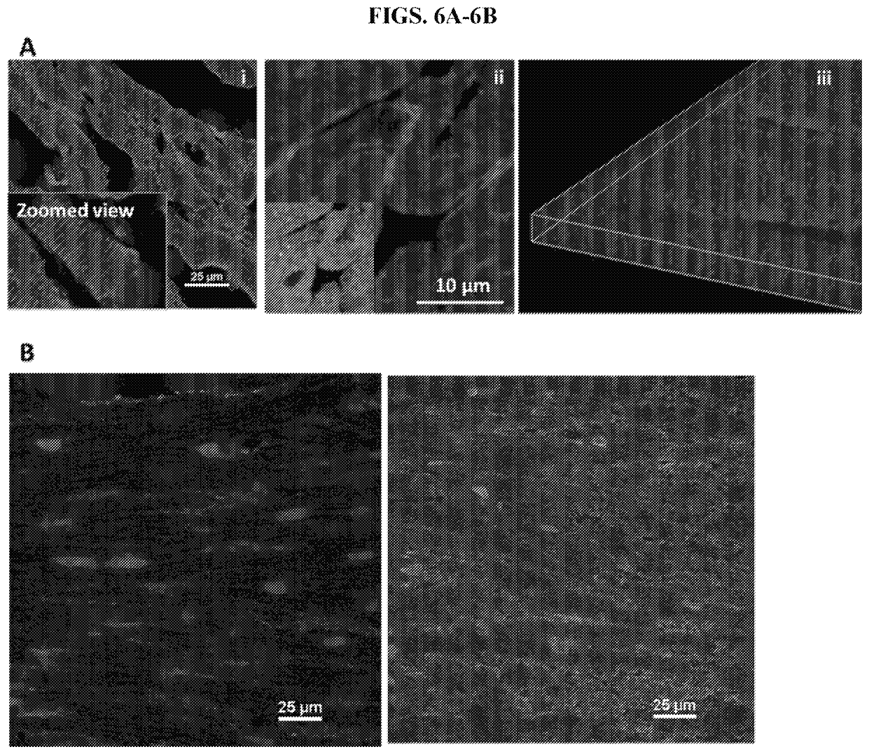

Independent of their utility for sorting iCM, antibodies to LSMEM2 may be useful for analyzing human and mouse heart tissue, where they specifically bind to TNNT2-positive cells (cardiomyocytes) in adult heart from both species (FIG. 6). These data are consistent with the fact that mouse and human sequences for LSMEM2 are identical for the epitope region for Ruby 2 antibody.

### Example 4—LSMEM2 as a Cardiomyocyte Biomarker

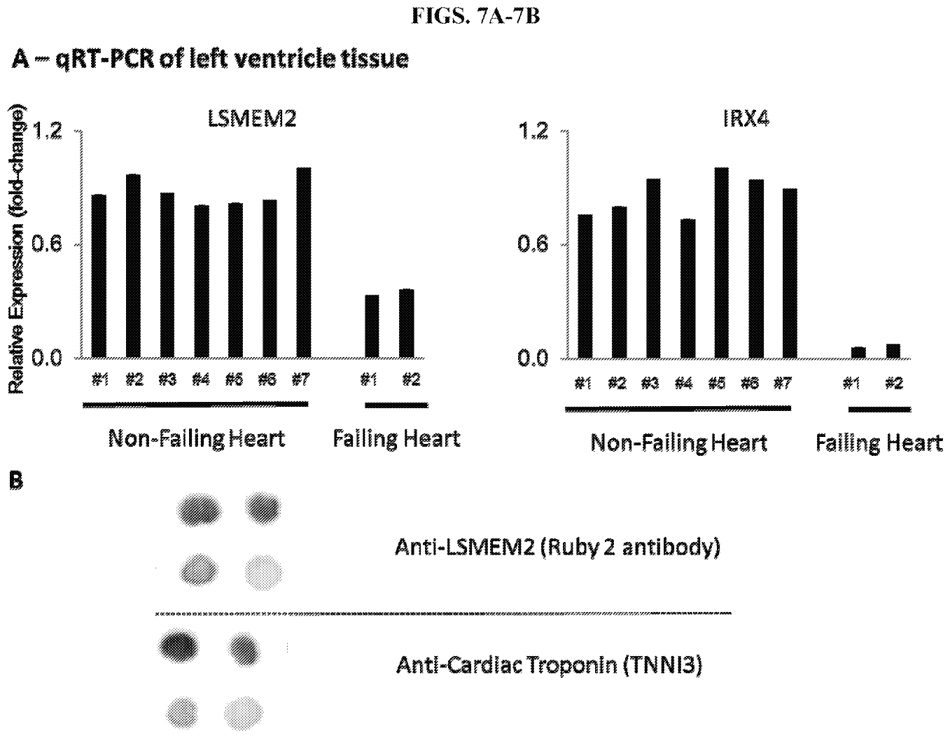

Cell surface proteins are often shed and appear as informative circulating biomarkers in numerous diseases including cancer and heart disease (Palmer et al., (2008) PLoS One 3, e2633; Wojtalewicz et al. (2014) PLoS One 9, e90461; Eleuteri et al., (2014) Biomarkers 19, 214-22). They may also be present in exosomes and microvesicles shed from the plasma membrane of cells and detectable in bodily fluids such as blood, plasma, serum, and urine. This class of biomarker can be especially informative in cases where the protein is unique to a single cell type in the body. This can be exploited as a biomarker in multiple ways. In one embodiment, development of new therapeutics will be dependent on the characterization of whether LSMEM2 is shed as a result of an enzymatic cleavage process and whether this process has a role in the causation, progression, or inhibition of disease. To support these possibilities, we have found that in human failing heart, mRNA levels of LSMEM2 are decreased compared to non-failing control, consistent with the potential ability for measuring LSMEM2 levels to predict a loss of cardiomyocyte mass (FIG. 7A). Second, we find the extracellular domain of LSMEM2 is present and detectable in serum from patients who have cardiac ischemia (FIG. 7B). Therefore, LSMEM2 may be a biomarker of cardiomyocyte health that could be detected earlier than troponins, which require cell death prior to their release into circulation. LSMEM2 may also be more prognostic and specific to cardiomyocytes than other currently used biomarkers for cardiac injury (e.g., CK-MB, BMP).

### Example 5—LSMEM2 as an Imaging or Therapeutic Delivery Target

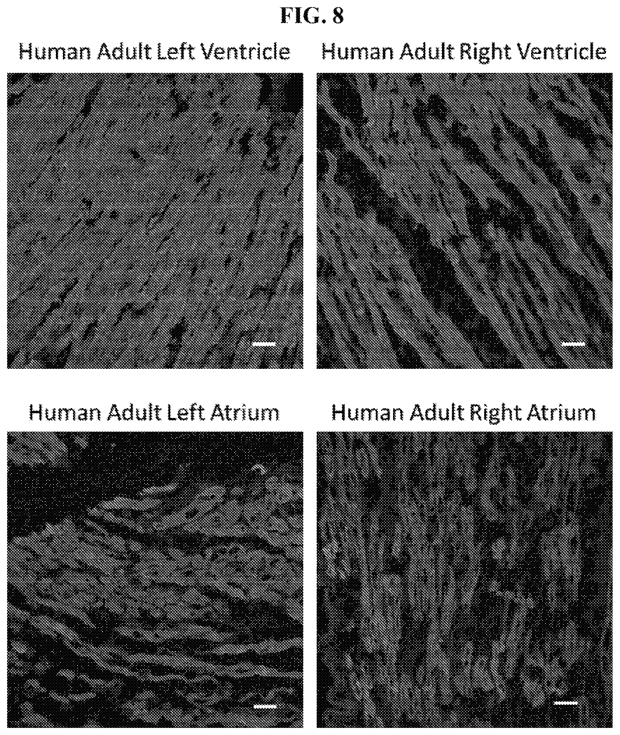

In the context of advanced heart failure and myocardial recovery with ventricular assist device (VAD), the ability to predict which heart failure patients will: i) respond to medical therapy alone; ii) progress to require VAD as a bridge to recovery; iii) benefit most from VAD; or iv) have little potential of recovery, would present new opportunities to refine treatment strategies. Also, the process of quantifying myocardial recovery in the clinic is not yet well-defined, but often includes cardiac catheterization, echocardiography, and cardiopulmonary/metabolic stress testing. Thus, it would be invaluable to develop new, less-invasive strategies to provide a quantitative measure of recovery and assist physicians in deciding if and when VAD explanation may be considered. Specifically, markers informative for metabolically-healthy cardiomyocytes could help overcome limitations of current markers that are merely indicative of perfusion or fibrosis (e.g. gadolinium). Cell surface proteins are particularly well suited to addressing these needs, as they can be exploited for use in real-time patient imaging (e.g., immuno-PET) (Wright et al., Soc of Nucl Med, 2013, 54(8):1171-1174). for tracking myocardial mass and structure and for histological analysis of tissue removed at time of VAD implantation. In this example, LSMEM2 antibodies could be used for live imaging of cardiomyocytes. Cell surface proteins can be also exploited as accessible markers of live cells for targeted drug delivery (Corrigan et al., Annals of Pharmacotherapy, 2014, 48(11):1484-1493; Newland et al., Pharmacotherapy, 2013, 33(1):93-104). In this way, LSMEM2 may be targeted using these antibodies, or others, to deliver pharmacotherapies to the cardiomyocyte, for example to preserve myocytes health or stimulate proliferation or growth. As shown in FIG. 8, anti-LSMEM2 antibodies specifically bind to human cardiomyocytes in each of the four chambers of the heart, supporting the possibility that LSMEM2 can be targeted in vivo using imaging or targeted drug therapy applications.

We claim:

1. A monoclonal antibody or antigen binding fragment thereof that is capable of binding an epitope of a LSMEM2 polypeptide having the amino acid sequence of SEQ ID NO:2.

2. The monoclonal antibody or antibody fragment thereof of claim 1, wherein the epitope comprises an amino acid sequence selected from the group consisting of SEQ ID NO:4, SEQ ID NO:5, SEQ ID NO:6, SEQ ID NO:7, and SEQ ID NO:8.

3. The monoclonal antibody or antibody fragment thereof of claim 1, wherein the epitope consists of an amino acid sequence selected from the group consisting of SEQ ID NO:4, SEQ ID NO:5, SEQ ID NO:6, SEQ ID NO:7, and SEQ ID NO:8.

4. The monoclonal antibody or antigen binding fragment thereof of claim 1, wherein is the monoclonal antibody is selected from the group consisting of a chimeric antibody, a recombinant IgG (rIgG) antibody, a diabody, a single chain antibody, a multispecific antibody, and a humanized antibody.

5. (canceled)

6. A method for isolating human cardiomyocytes from a cell mixture derived from human pluripotent stem cells, wherein the method comprises contacting to the cell mixture the monoclonal antibody or antigen binding fragment thereof of claim 1; and recovering antibody-bound cells from the contacted cell mixture, wherein the recovered cell population is a substantially homogeneous population of human cardiomyocytes.

7. A method for separating a cell population containing human cardiomyocytes from a cell mixture derived from human pluripotent stem cells, wherein the method comprises contacting to the cell mixture the monoclonal antibody or antigen binding fragment thereof of claim 1; and separating antibody-bound cells from the contacted cell mixture, wherein the separated cell population is a substantially homogeneous population of human cardiomyocytes.

8. A method for detecting human cardiomyocytes in a cell mixture from human pluripotent stem cells, comprising contacting to the cell mixture the monoclonal antibody or antigen binding fragment thereof of claim 1; and detecting the antibody.

9-10. (canceled)

11. A method for predicting, diagnosing, or monitoring a cardiovascular injury in a subject, the method comprising
measuring the level of expression of LSMEM2 in a biological sample obtained from the subject; and
comparing the level of LSMEM2 with the LSMEM2 level from a control sample, wherein a measured level or characteristic of LSMEM2 that is different than the control level or characteristic is indicative of a cardiovascular injury.

12. The method of claim 11, wherein the biological sample is selected from the group consisting of whole blood, a blood fraction, plasma, serum, urine, and heart tissue sample.

13. The method of claim 11, wherein the cardiovascular injury is selected from the group consisting of myocardial infarction, cardiac ischemia, acute coronary syndrome, cardiomyopathy, heart failure, cardiac remodeling, and cardiac dilation.

14. The method of claim 11, wherein the biological sample is serum and the cardiovascular injury is cardiac ischemia.

15. The method of claim 11, wherein the measuring comprises contacting a LSMEM2 binding agent to the sample; and (b) measuring the level of LSMEM2 bound to the binding agent.

16. The method of claim 15, wherein the binding agent is an antibody or antigen-binding fragment thereof.

17. The method of claim 15, wherein the binding agent is the monoclonal antibody of claim 1.

18. The method of claim 15, wherein the antibody or antigen-binding fragment thereof is immobilized on a solid support.

19. The method of claim 11, wherein the level of LSMEM2 is measured using an assay selected from RIA, ELISA, mass spectroscopy, fluoroimmunoassay, immunofluorometric assay, immunoradiometric assay.

20. (canceled)

21. A method for detecting, diagnosing, or monitoring myocardial recovery in a subject, the method comprising
measuring the level of expression of LSMEM2 in the subject or in a biological sample obtained from the subject; and
comparing the level of LSMEM2 with the LSMEM2 level from a control sample, wherein a measured level that is different than the control level or characteristic is indicative of a myocardial recovery.

22. The method of claim 21, wherein the measuring is performed non-invasively.

23. The method of claim 21, wherein the biological sample is selected from the group consisting of whole blood, a blood fraction, plasma, serum, urine, and heart tissue sample.

24-29. (canceled)


SEQUENCE LISTING (8 sequences)

SEQ ID NO: 1 - DNA, 10200 bp, Homo sapiens
cgagatcgccccactgcactccagcctgggcaacagagcgagactctgtctcaaagaaaagaaaactgggtgatgtggaaaagaatgtacacagaaggcctcactgaaggggtggcatttgagttcgctgacaagacggggctggccacagggctatctgggagcagagcattccaggagggaacagccaccaccaagcccctgaggtggtaacaagcttgttgactttgaggaacatcagggaggccagtgtgagacaacggagtgacaggtttgaggtgtgtaggaagtagagcccacagtcttgctttgcccccactgggaaggaagggcacagggatccaaccacagagggaaagctggaagctggaccacctcctcctttcaaggtaagcagtgatgcgcatccttacacaaccttatgggacctgtctgcccttgtggagatgggagcaggggaagaccaaaaccgtggattgtgctaaatgctccagatgttgttctcagtgtacactgccagggaggagccactgttattccgactttacacatgagcaaactgagttcagagaggggagaccctttacctaaggttgcaagcttagtaacaaatgaaccagcctccaacctaggtccacccactggccagcatgggtgctgttggtgtatctgcccacttgtgaagctatcagagccctcatggcattgggattcagttgtccatcaccctctataagacacacacaacaatagcattgtgtgctagcaggtcatgcagtggaggctgctgtgtttgctgggaggagagatgactgggctgtgggtaggagtctgcaggggtagaggggtggccaaatagagatgcacaggcttcctttgtctgtcttggctgccacatggggccttgctgagcctggagtggccattctggatgtatgggggaggtgtgcttccccttgaaggtccctgggcacctggactagtggaggcaggctcctggctggctccctcaccaggtagatagttcaggctggcccacgtggatcactctgccccttcccatggctgagggctgccccctctaaatatagcccagtgggctgagctcaggcctattttaggccaaggctgggaggcagccaggccttccctgggctcacaaaggagccactgctgcatttgtccagtcctgctactggatgccatcattggcccccgactgcccactgcttgccatgcctgaggagacccaagaaggtaggagagggtgaccatgagggagggcaaggcctcagtcctgtgaccttgtggaccatggagagacccaccctctgtcccactgacctcactgtcttgagtagttagttacccattttcatgctccagctccagtaaaaggacctgccccctgtgcagggaggaaaagcctgccatgcctggagccccttgtcacctcaggccctgcttcagtttcccctgctggggacatcaaggagggatgggagaagctacccagttaggagtgggtcaactacaggctgtgctcaggtgggtgtgggcatccggtcagctgcctggccggttcagctcttaaggaaggaagctgggattggggtgggtagtggttccagaaagcatttgggtacctgtcccatggactgaaaagtagttaggcctctggcctcccctgacttgggtctaaatatccccaactgcctagggttgggggtcccttggcctggtcagacagagactcagctagtcaagagcttggtggccactcacctaactgcaccactgcatataagaagtactgatgctcagaggggccagggcagcaggaatctaggaagctacaggtccagtactggccaggacgtaggactgtctgctccatggctccatgttatggcccatgacagagcatggcccagctagcaagctcctcttatcacattttcaactttttttttttttttgagatggaatctcgctctgtcacccaggccggagtacagtggcgtgatctcggctcactgcaacctccgcctcccagattcaagcaattctctgcctcagcctcccaagtagctgggattacaggcgcctgccaccatgcctagctaatttttgtatttttagtagagacgggggttcaccatcttggccaggctggtcttgaactcctgacctcgtaagccacccacctcagcctcccaaagtgttgggattacaggggtgagccaccgcgcctggcctcaactttttttttttttgtcacccaggctggagtgcagtggtgcggtctcggctcactgcaacctctgcctcccagattcaagctattctcctgtctcaggctcccaagtagctgggattacaggcatgtgctaccatgcccggctaatttttgtatttttagtagagacaaggtttcaccgtgttggccaggctggtctcaaactcctgacctcaggtgatccacccaccttggcctcccaaattgttgggattacaggcgtgagccactgccaacattttcaacattgcaggagaggggccttgcccctggtagggggaaatgtttactttcatgcatgtgtgtgtgtagagaagggtctccctggtcttgaactcctggcctcaaccaatccaatcctcccaccttggcctttcttttcttttttttttttttttgagatggagtctcgctctgtcgcccaggctggagtgcagtggcgcgatctcagctcactgcaagctccgccttccgggttcacgccattctcctgcctcagcctcctgagtagctgggactacaggagcccgccactacgcctggctaattttttgtatttttagtagagatggggtttcactgtgttagccggcatggtctcgatcttctgacctcgtgatccacccacttcggcctcccaaagtgctgggattacaggtgtgagctaccgcgcccggccagccaccttggcctttcaaagtgctgggattagaggcataagccactgtgcctaccctcttttgggatcttaacaataacatttactgtgtgtttttcctgtaataaacaattcacattcaatttagaaagtgtgagaaaagcacaacaaagtacaaagagggaaatttttttttttttttgagacagagtcttgctctgtcgcccaggctggagtgcagtggcacgatcttggctcattgcaagctctgcctcccgggttcatgccattctcctgcctcagcctcccgagtagctgggactacaggcgccagccaccatgcccagctaatttttgttttgaattttttagtagagacggggtttcactgtgttagccaggatggtctcgatctcctgacctcgtgacctgcccacctcggccttccaaagtgctgggattacaggcatgggccactgcgcccggcccttttttttttttttttttctgagacggagtcttgctctgtcgccccgggtggagtgcagtggcacgatctctgctcactgcaacctctgcctcccaggttcaagtgattctcctgtctcagcctcccaagtagctgggactacaggcgcctgccaccatacctggctaatttttaaatatttttggtagagacggggtttcaccatattggccaggctggtctcaaactcctgaccttgtgatccacttgccttggcctcccaaagtgctgggattataggtgtgagccactgcgcccggcagcagggaatttttttttttttttttttttttttggagatagggtctcactctgttgcccaggctggagtacaatggcatgatcacagctcactgcagccttgacttcctggactcaagcaaccctcccacctcagcctcctgagaagctgggaccacagaagcatgccaccacaccaaactaatctttaattacttttttgttttttgagacgaggtctagctctgtcgcccaggctggagtgcagtggcttgagcttggatcactgcaagctccgcctcctgggttcaagctattctcctgccttcagcctcctgagtagctgggactacaggcgcccatcaccatgcccggctaattttttgtatttttagtacagatggggtttcaccatgttgaccaggctgatcttgaactcctgacctcgtgatccgcccacctcagcctcccaaagtgctgggattacaggtgtgagccaccatgcccggccctaatctttgatttttttgtagagatggggtctccctacgttgccctggctggtttcaaactcttagactcaaatgatcctcctgccttggcctcccaaagtgctgggattacaggcatgagccacagtgcccggcccaaagagagaatttaatatgaccttcaacccaccacccagacataatgactggcagcacttgcttgtttttgttgtacttgcttttgagcacgtcaagaggcagtttcaagtaaatcatggaggttgagttactgtcctgagaatgacagtcccagagtgcaaggccacccttgctcctatctggaggtgtcccctcaatgctcttccctactgttatcagcttccaggtgggtatctggagctcttggcatctggaccaggatttttagacatcaagttaaagaacaactagaagaaaatataattaaatattggccaggtacggtggctcatgcctgtaaacccagcactttgggaggacaagatgggcagatgacttgaggtcaggagttcaagaccagcgtggccaaaatggtgaaaccccagctgtactaaaaatgcaaaaattagctgggcatggtggcacgtgcctgtaatcccagctactggggagacgaggcaggaaaatcacttgaacccaggaggcagaagttgcagtgagctgagatcatgccactgtactccagtctgggtgacacagtaagactctgtctcaaaaaaaacagacaaacaaaaggccaggcatggtggctcatgcctgtaatcccagcactttgggaggctgaggtgagcagatcacgaggtcaagagatcgagaccatcctggccaacatggtgaaaccccgtctctactaaaaatacaaaaattagccaggcgtggtggcacgtgcctgtagttccagccactcgggaggctaaggcaggagaatcggttgaacctgggaggcggaggttgcagtgagctgagattgcgccactgcactccagcatggcaagagagggagattctgtctcaaaaacaaacaaacaaacgaaacaccggctgggtgcgatggctcacgcctataatcccagcactttgggaggctgaggcgggtggattacctgaggtcaggagttcaagaccagcctggccaagaaggtgaaaccccatctttatcaaaaaatacaaaaattggccgggcgcggtggttcacacctgtaatcccagaactttgggaggctgaggcagacggatcacaaggttaggagatcgagactatcctggctaacatggtgaaaccccatctctactaaaaatacaaaaaattagccgggcatggcagcggacgcctgtagtcccagctacttgggaggctgaggcaggagaatggcacgaacctgggaggcggagcttgcagtgagccgagatcgtgccactgcactccagcctgggcggcagagcgagactccatctcaaaaaaaaaaaaaaaaaaaaaaatttgcagggtgtggtggtgcacacctgtagtgctagctactaaggagggtgaggtaggagaaacccttgaacccgggaggcggaggttgcagtgagcccagatcgtgccactgcactccagccagggcaacagagactctgtctcaaacaaacaaacaaaaagcaaaaacttcaatgagtgtgaaagtacataattaaaatcaaaagaaaagtacacagacccaagtgactaaatgggcagaggcttcaaattgataattaagaagccacacacagctgggcatggtggcttgcgcctataatcccagcactttgggaggccaaggcgggtggatcacttgaggtcaggagtttgagaccagcctggccaacatggcaaaacctggtctctactaaaaatacaaaaattagccaggcatggtggcaggtgcctgtagtcccagctacttgggaggctgaagcacgagaattgcttgaacctgggaggtggaggttgcagtgagctgagatcgcaccactgcactccagcctgggcaacacagcaagactgtctcaaaaaaaaaaaaaaaaaaaaaagcgccacacacgtattagtcacactccctaggctaagaaacccaataaaataagaaagaccatttctcatctctcaagttagcaaatatatgtgtgcacgtgtgcatgtaaatataatttttttcctatggggatgcctaatggtggcaatattataccctgcgggggagctgtggacagtgcttctggaggacagtttggtaatagctttaaaaaaaatcaggccgggcatggtggctcacacctgtaatcccagcattttgagaggccaagcaggatggatctcttgagaccaggagctcgagaccagcctgggcaacatagtaaaaccctgtctctacaaaaaatacaaaaaaaattaactgggcatggtagtgcatgcctgtagtcccagctactgggaaggctgatgcaggaggatcacctgagcccaggaggtggaggctgcggtgaactgtgatcactccactgaactccagcctgggtgaaagagtaagaatctgtctcaaaaacaaaacaaaaatctcaggccaggcgcgatggctcacgcctgtaatcccagcactttgggagtctgaggcgggcggatcacttgaggtcaggagtttgagaccagcctagccagcatggtgaaacctggtttctactgaaaatacaaaaattagcagggcgtggtggcacacatctgtaatcccagctacttgggaggctgaaacaggagaatcacttgaacccaggaggtggaggttgcaatgagccaagattgcgccactgcactccagcctgggcaacagagcaagactctgtctcaaaaacaaaaaaacaggccgggcacggtggctcacgcctgtaaatcccagcactttgggaggccgaggtgggtggatcacaaggtcaggggttcaagactagcctggccaagatggtgaaaccccatctccactaaaaatataaaaagtagctgggtgtggtggaaggcgcctgtaatcccagctactcaggaggctgaggcagagaactgattgaacccgggaggcagaggttgcagtgagccgagatcttgccattgcactccagcctgggcgacagagggagactccatcttaaaaaaaccaaaccaaaacaaaaaacgaccaaatgcagtggctcatgcctgtaaatgccaggactttggaaggccgaggcgggcagatcacttgaggtcaggagttccagaccagcctggccaacatggtgaatcctcatctctaccaaaaatacaaaaattagctgggcgtggtggcacacgcctgtaatcccagctactcaggaggctgatgcaggagaatcgcttgaacccaggaggtggaaggtgcagtgaggcgagatcacgacatagcactccagcctgggcaatacagggagactccatctcaaaaaacaaacaaacaaacaaaaatccctgctcttggatccagtaattccacttctgggaattaaactatcaggaaattgggaaaaaaccttcatattcaacggtggatgtcctgggggttaatttcaaaagggaactgcagcggtgagccaagatcgcgccattgcgctccagcctgggcaacaagagcgaaactccgtctcaaaaaaaaaaggggggcggggactgcaaacaacctagatgcccaggcagcctcagggagatgtgtgaagggagagacctcagaggcatgtgcaacagtataacaaagaagggtctccccaacagtaaatgttgaatgaaaacagcagggttcacacatgtacagcagagtcacactgagtatgttaacaaacacagaaccaaggccaggagggaaaggcaccagagtggctacaatagggctttctgaccagaagatgccaggcattgggtctgcagggagaactgacaaaagaggcaggactgaattccatttgctgcaggtttagcattcaaggctgataaactgctagaaataagcaggcagaggaatgcctgagtgagtccctggggtgcttggaaggaaacaaatcaaatgtacctgttgctcctgtatacagaataagcctgtaagggtctgggatcatcctggatccaaatcaagggtaattcctgagtcagttttaggggatttggaaccaaatgtcccactatccctatcatccgcaagtccttgctgcctgaagccacctggtagaaggaagggggttgacccaccactggctaaatcagcctgcgctgcccctgcagactccgtggcgccaatgatgcccagccagaggagcagggggccattggcccccaaccacgtgcatgaggtatgcctgcaccaggtggagtccatcagcgacctacatagtggaggtgagtggggacagtggggtggatgatgtgctgggggaggggacgaaagcaggtgcacccaccaccctcccaatgtcccatccacccagcaggcacactgcgcccctatctaactgaagaggcacgaccgtgggatgagctgctgggcgttttgccgccgtcactgtgtgcccaggctggctgcagccctgtgtacagacgaggagggttcctgctgctgctcgcgctgctggtgctcacttgcctagtgctcgcactcctggctgtctacctgagcggtatggacgcatagggtgctagtaggaatggaaagcaaggcagtggggactgggaggcccttctgtgcagctggaaggagtaactgcagatgctgcagcagtgaaggatggggcgggaggcccgtggctgcccctggtcctgggctgggtctgcaggggtcacggggtggcacatggtctgatgatcccccacttcccattcacagtgctgcagagtgaatccctgcgcatcctggcacacacgctccgcacgcaggaggagacactactcaaactccgcttggccagcctcagccagcttcggaggctcaactccagtgaggcccaagcacccagctgagatgccatttggatctggggcctgggggtgtgtgttggtgggggaataaagtggctatgggcaggtctctccttccctactgctggctgccacatctacactatttccttggtgagatttttgtacaagaacctgttgttaacttaatggctgcctccctctcctgatctcttcaagccaggcctagccaagcctctggtgccaaagcctcgctttgggtggcccaaggtcaggggaaggggcaccagcctcctggctcctcctgtggcctgtcaacactctctggccaccccagatggcaggttctgaagtctagaaatagctgcccccatagcaatcacccaaactctgccagggctcccagtgagggtccagcctcagctcttaaagacaggctgggctctgagtagggggagaggcaccacctggggaagactgccttggagggcaaaggtgacatcgtggaagaccagttgggcccaagcctgggatagtagatggggagggagaacagaaaaatacccaggggttaagaccacagaaggctgaccgttctgtttaattatctgtaataatccttaaaaatcagcacacaaatccattccaggtagctttgccaccccacctgctgtggtggcatttgtccagatgccaccacccccctaagagtgggtcatcctgggggagcaaggcctgagaaaggaaaggaagggaaaggccccctagtgcctgccccacagccctgaggaaggcacatatttagcctggcctgagacaggacaggaaggggccacagggctgagtgcagggacaaaggggtcagggtcccaagtgtccgggcccccagctacccaccccatgtctgtttttggttttgtcattaaaaaaaataaagtgacaaatactggtgga

SEQ ID NO: 2 - protein, 164 aa, Homo sapiens
MPSLAPDCPLLAMPEETQEDSVAPMMPSQRSRGPLAPNHVHEVCLHQVESISDLHSGAGTLRPYLTEEARPWDELLGVLPPSLCAQAGCSPVYRRGGFLLLLALLVLTCLVLALLAVYLSVLQSESLRILAHTLRTQEETLLKLRLASLSQLRRLNSSEAQAPS

SEQ ID NO: 3 - protein, 47 aa, Homo sapiens
  MISC_FEATURE: extracellular domain
YLSVLQSESLRILAHTLRTQEETLLKLRLASLSQLRRLNSSEAQAPS

SEQ ID NO: 4 - protein, 12 aa, Homo sapiens
  MISC_FEATURE: Ruby1
RTQEETLLKLRL

SEQ ID NO: 5 - protein, 12 aa, Homo sapiens
  MISC_FEATURE: Ruby2
QEETLLKLRLAS

SEQ ID NO: 6 - protein, 12 aa, Homo sapiens
  MISC_FEATURE: Ruby3,4
QLRRLNSSEAQA

SEQ ID NO: 7 - protein, 12 aa, Homo sapiens
  MISC_FEATURE: Ruby5,6,7,8
RILAHTLRTQEE

SEQ ID NO: 8 - protein, 12 aa, Homo sapiens
  MISC_FEATURE: Ruby9
ESLRILAHTLRT